(19) United States
(12) Patent Application Publication
(10) Pub. No.: US 2025/0188489 A1
(43) Pub. Date: Jun. 12, 2025

(54) CONJUGATION OF ADENO-ASSOCIATED VIRUSES

(71) Applicant: TAVIRA THERAPEUTICS BV, Heverlee (BE)

(72) Inventors: Els HENCKAERTS, Leuven (BE); Benjamien MOEYAERT, Leuven (BE); Tine BROUNS, Leuven (BE)

(73) Assignee: TAVIRA THERAPEUTICS BV, Heverlee (BE)

(21) Appl. No.: 18/842,153

(22) PCT Filed: Apr. 28, 2023
(86) PCT No.: PCT/EP2023/061210

§ 371 Date: Aug. 28, 2024

(30) Foreign Application Priority Data
Apr. 28, 2022 (EP) 22170610.4

(51) Int. Cl. C12N 15/86; C07K 14/005

(52) U.S. Cl. CPC C12N 15/86; C07K 14/005; C12N 2750/14122; C12N 2750/14143; C12N 2750/14145; C12N 2810/859

(57) ABSTRACT

The invention relates to an adeno-assisted virus (AAV) VP1, VP2 or VP3 capsid protein characterized in that in one or more of the VP1-VP2 transition region, VR-I region, VR-IV region and VR-VIII region a sortase recognition is inserted, wherein n and m range from 0 to 25, and wherein X is any natural amino acid.

### FIELD OF THE INVENTION

The present invention relates broadly to the field of gene therapy. Particularly, the invention relates to a technology for conjugating heterologous molecules such as targeting molecules to AAV capsids in a controlled manner.

### BACKGROUND OF THE INVENTION

Gene therapy (GT) is a therapeutic modality that involves modifying cellular physiology through the addition of a transgene, thereby treating or preventing disease. This innovative technology uses viral vectors to correct or replace the faulty genes that cause disease. Gene therapy holds great promise for the treatment of rare and more complex devastating diseases for which currently no curative treatment is available.

Adeno-associated virus (AAV) is a small, non-pathogenic virus that is currently the prime gene delivery vehicle in gene therapy. It has a broad tissue tropism, is not associated with a disease phenotype and not highly immunogenic. Moreover, AAV has the potential to provide a long-lasting therapeutic effect after single-dose administration.

Recombinant AAV (rAAV) is an engineered version of the virus whereby the viral genes are placed in trans and replaced by a transgene of approximately 4.5 kb. Though rAAV-based GT products in general have shown to be safe and efficacious, they do suffer from several drawbacks. For instance, the processes underlying transduction, i.e. cellular entry at the target tissue, uncoating and expression of the viral particles' therapeutic payload inside target cells are inefficient, therefore necessitating large vector doses to achieve therapeutic effect. After systemic administration, this can lead to accumulation in the liver and an immune response to the therapeutic vector. Importantly, high doses of rAAV have been associated with severe adverse events such as hepatotoxicity and thrombotic microangiopathy and resulted in the death of several patients in AAV GT trials. Additionally, depending on the AAV serotype, 40-70% of the human population carries AAV neutralizing antibodies. These antibodies contribute to the observed immune response, reduce efficacy and prevent re-administration of the GT product, leading to seropositivity being an exclusion criterion in many GT trials. Next to these biological issues, the production of large quantities of highly purified rAAV products is expensive and production capacity is limitingly scarce.

One of the most promising approaches to solve these issues is the design of novel capsids that display improved immune evasion and transduction properties. By narrowing the broad tropism of naturally occurring AAV vectors to specific tissues or cell types through capsid engineering, the rAAV product becomes more potent, allowing treatments at lower doses. This reduces the safety risks and the manufacturing costs. However, despite the significant efforts in capsid engineering (e.g. directed evolution capsid design platforms), no ground-breaking innovations have been validated in the clinic. This is in part due to the broad tropism of AAV serotypes found in nature that are used as starting material for directed evolution library generation, the difficulty in production of novel rAAV serotypes and the need for screening of capsid libraries in rodents and non-human primates (NHPs), the latter being a roadblock to human translation.

A promising method for engineering capsids with increased specificity for target tissue and lower off-target transduction without affecting manufacturability, is conjugating AAV particles with specific (re)targeting groups at a post-manufacturing stage. Several groups have genetically incorporated targeting groups into the capsid proteins, or conjugated them with adaptor molecules that allow for covalent or non-covalent linkage after the AAV particles are purified. These rAAV conjugates display higher transduction efficiency of specific tissues, and have lower off-target effects, e.g. in the liver. However, the required modifications to the capsid often affect the production yield. In addition, the low modularity and complex, often multi-component genetic or chemical design setup render these systems largely incompatible with current commercial manufacturing practices. More recently, capsid proteins have been genetically fused with nanobodies which resulted in improved targeting. However, the targeting efficiency was heavily dependent on the specific AAV-nanobody construct. This can be traced back to unpredictable folding and quaternary structure formation of the modified AAV virions and an unknown effect of nanobody fusion on the supramolecular dynamics caused by direct genetic integration of the nanobody. Also, the genetic insertion of large proteinaceous moieties negatively impacts the efficiency of affinity purification using commercially available resins.

In summary, conjugating AAVs with targeting groups is a promising strategy for improving the transduction profile of rAAV particles for gene therapy, however current methods lack the design and/or performance qualities needed for a commercial gene therapy product and are faced with steep manufacturing hurdles. In order to become a clinical and commercial success, better approaches for AAV conjugation are direly needed.

### SUMMARY OF THE INVENTION

Through extensive experimentation, the inventors have found that sortase recognition sequences can be introduced internally in capsid sequences of AAV particles to enable conjugation of targeting molecules with the AAV capsids to produce conjugated AAV particles and to more efficiently and specifically target tissues and cells. Surprisingly and in contrast to the general consensus in the technical fields of molecular biology and biotechnology wherein sortase recognition sequences are typically used C-terminally, the inventors have found that insertion of a sortase recognition sequence in the VP1-VP2 transition region, the VR-I region, the VR-IV region, and the VR-VIII region maintains structural integrity of said region(s) after being subjected to a sortase reaction. The use of an internal sortase recognition sequence is particularly valuable for generating a new “generation” of tailor-made AAV particles with improved properties, for example a more efficient and specific targeting of tissues and cells of interest. In addition, the molecular design considerations made by the inventors allow for an increased modularity of AAV production methods and platforms, whilst maintaining similar production efficiencies when compared to the appropriate parental AAV serotype.

Accordingly, one aspect of the invention provides an adeno-associated virus (AAV) capsid protein characterized in that in one or more of the VP1-VP2 transition region, VR-I region, VR-IV region and VR-VIII region a sortase recognition sequence is inserted. In certain embodiments, the sortase recognition sequence has a general sequence motif selected from the group consisting of Xn-LPXTG-Xm [SEQ ID NO: 10], Xn-NPXTG-Xm [SEQ ID NO: 40], Xn-LPXTA-Xm [SEQ ID NO: 41], Xn-LAXTG-Xm [SEQ ID NO: 42], Xn-LPXAG-Xm [SEQ ID NO: 48], Xn-LPXLG-Xm [SEQ ID NO: 49], Xn-APXTG-Xm [SEQ ID NO: 50], Xn-LPXSG-Xm [SEQ ID NO: 51], Xn-FPXTG-Xm [SEQ ID NO: 52], Xn-XPKTG-Xm, [SEQ ID NO: 53], and Xn-LPEXG-Xm, [SEQ ID NO: 54], wherein n and m range from 0 to 25, and wherein X is any natural amino acid independently selected for Xn and Xm. Preferably, the (AAV) VP1, VP2 or VP3 capsid protein characterized in that in one or more of the VR-I region, VR-IV region and VR-VIII region a sortase recognition sequence Xn-LPXTG-Xm [SEQ ID NO: 10] is inserted, wherein n and m range from 0 to 25, and wherein X is any natural amino acid. Optionally, n and m range from 0 to 20. Preferably, X is Glutamic acid (E) or Glutamine (Q).

In certain embodiments, the VP1-VP2 transition region is defined by SEQ ID NO: 1 [PVKTAP], the VR-I region is defined by SEQ ID NO: 2 [SSQSGASN], the VR-IV region is defined by SEQ ID NO: 3 [SRTNTPSGTTTQSRLQFSQAGASDIRDQS], and the VR-VIII region is defined by SEQ ID NO: 4 [QYGSVSTNLQRGNRQAATADVNTQGV] in AAV2 or a corresponding amino acid sequence in another AAV serotype.

In certain embodiments, the insertion in the VR-IV region is in the fragment with SEQ ID NO: 5 [TPSGTTTQS] and/or the insertion in the VR-VIII region is in the fragment with SEQ ID NO: 6 [LQRGNRQAA] in AAV2 or a corresponding amino acid sequence in another AAV serotype.

In certain embodiments wherein in a region a sortase recognition sequence LPXTG [SEQ ID NO: 10] is inserted, one or more amino acids of said region are deleted, and/or one or more amino acids of said region are substituted.

Optionally, the AAV is AAV2 or AAV9.

Optionally, n is between 15 and 20, or between 10 and 15, or between 5 and 10, or between 0 or 5, or is 10, 9, 8, 7, 6, 5, 4, 3, 2, or 1. Preferably, n is from 5 to 20, preferably from 5 to 15, more preferably from 10 to 15.

Optionally, m is between 15 and 20, or between 10 and 15, or between 5 and 10, or between 0 or 5, or is 10, 9, 8, 7, 6, 5, 4, 3, 2, or 1. Preferably, m is between 5 and 20, preferably between 5 and 15, more preferably between 10 and 15.

In certain embodiments, the linker sequence Xn or Xm consists of at least 80% of Glycine (Gly), Serine (Ser), Threonine (Thr), and Alanine (Ala). Preferably, the linker sequence Xm or Xn consist of amino acids selected from Glycine (Gly), Serine (Ser), Threonine (Thr) and Alanine (Ala).

In certain embodiments, X in LPXTG is Aspartic acid (Asp), Glutamic acid (Glu), Asparagine (Asn) or Glutamine (Gln), preferably Glutamine (Gln).

In certain embodiments, one or more Lysines (K) of the AAV capsid protein are mutated into Glycine (Gly), Serine (Ser), or Alanine (Ala).

A further related aspect of the invention is directed to a nucleic acid encoding the capsid protein according to any one of the embodiments described herein.

Yet a further related aspect of the invention is directed to an expression vector comprising the nucleic acid of the preceding aspect.

Further related aspects of the invention are directed to AAV particles. Particularly, the AAV particles comprise AAV capsid protein having a sortase recognition sequence inserted in one or more of the VP1-VP2 transition region, VR-I region, VR-IV region and VR-VIII region. Both AAV particles prior to a sortase conjugation reaction and after a sortase conjugation reaction (i.e. conjugated AAV particles) are intended. Thus, a further aspect of the invention is directed to an AAV particle comprising an AAV capsid protein according to any one of the embodiments described above. Additionally, a yet further related aspect of the invention is directed to a conjugated AAV particle comprising an AAV capsid protein characterized by a remnant sortase recognition sequence (i.e. a modified sortase recognition sequence as present after a conjugation reaction) in the VP1-VP2 transition region, VR-I region, VR-IV region and/or VR-VIII region, and wherein the remnant sortase recognition sequence is operably linked to a heterologous conjugate molecule.

In certain embodiments, the conjugated AAV particle is operably linked (i.e. fused) via the sortase recognition sequence to a heterologous conjugate molecule, preferably wherein said conjugate molecule is characterized by the presence of a terminal triglycine amino acid sequence. Optionally, the heterologous conjugate molecule is a small molecule, carbohydrate, a lipid, or a polypeptide.

In certain embodiments, the conjugated AAV particle is operably linked (i.e. fused) via the sortase recognition sequence to a targeting moiety. Optionally, the targeting moiety is a ligand of cell receptor, or a protein binding to a cell surface protein. Optionally, the protein binding to a cell surface protein is an antibody or a nanobody. Optionally, the antibody or nanobody specifically binds HER2.

A yet further aspect of the invention is directed to the use of a conjugated AAV particle according to any one of embodiments described herein, as a medicament.

A related aspect of the embodiments of the invention described above is directed to methods of producing the conjugated AAV particles as described herein. In particular embodiments, the methods of producing a conjugated AAV particle, comprise the steps of
contacting cells with one or more nucleic acids encoding a AAV capsid protein wherein one or more of the VP1-VP2 transition region, VR-I region, VR-IV region or VR-VIII region comprise a sortase recognition sequence;
allowing the cell to assemble the plurality of AAV capsid proteins into an AAV particle and collecting said AAV particles therefrom; and
contacting the AAV particles with a sortase and a heterologous conjugate molecule.

The above and further aspects and preferred embodiments of the invention are described in the following sections and in the appended claims. The subject matter of the appended claims is hereby specifically incorporated in this specification.

### BRIEF DESCRIPTION OF THE DRAWINGS

FIG. 1. (A) schematic genetic buildup of wild type AAV (wtAAV) and recombinant AAV (rAAV). (B) Schematic representation of the Sortase A mechanism

FIG. 2: (A) Schematic overview of VP1, VP2, VP3 proteins, and VP1-VP2, VR-I, VR-IV, and VR-VIII regions. (B) General representation of an internal LPQTG motif flanked by linker sequences.

FIG. 3: The LPQTG motif was inserted in AAV2-HBO VR-IV (between AA 453 and 454) VR-VIII (between AA 587 and 588), without linker or flanked by 1 or 2 GGSGS [SEQ ID NO: 47] repeats at either side of the motif A band consistent with the molar mass of the N-terminal fragment of VP3 with conjugated Nb (˜19 kDa) is visible in VIII-1 and VIII-2, demonstrating a conjugation reaction. Srt-A: Sortase A only, Nb: nanobody only

FIG. 4: AAV2, VR-I, 0-1-2 L. AAV2-HBO with an LPQTG tag [SEQ ID NO: 43] located at position 265 with 0, 1 or 2 GGSGS [SEQ ID NO: 47] linkers flanking both sides. Arrow: conjugated product.

FIG. 5: AAV2, VR-IV, 0-1-3-4-5 L. AAV2-HBO with an LPQTG tag [SEQ ID NO: 43] located at position 453 with 0, 1, 3, 4 or 5 GGSGS [SEQ ID NO: 47] linkers flanking both sides. Arrow: conjugated product.

FIG. 6: AAV2, VR-VIII 0-1-2-3-4-5L. AAV2-HBO with an LPQTG tag [SEQ ID NO: 43] located at position 587 with 0, 1, 2, 3, 4 or 5 GGSGS [SEQ ID NO: 47] linkers flanking both sides. Arrow: conjugated product.

FIG. 7: AAV9, VR-IV 0-1-2-3L. AAV9-A with an LPQTG tag [SEQ ID NO: 43] located at position 455 with 0, 1, 2 or 3 GGSGS [SEQ ID NO: 47] linkers flanking both sides. Arrow: conjugated product.

FIG. 8: AAV9, VR-VIII 0-1-3L. AAV9-A with an LPQTG tag [SEQ ID NO: 43] located at position 589 with 0, 1, or 3 GGSGS [SEQ ID NO: 47] linkers flanking both sides. Arrow: conjugated product.

FIG. 9: Comparison of 5 distinct constructs. AAV2-HBO with an LPQTG tag [SEQ ID NO: 43] located at position 265 with 2 GGSGS [SEQ ID NO: 47] linkers flanking both sides, AAV2-HBO with an LPQTG tag located at position 453 with 3 GGSGS [SEQ ID NO: 47] linkers flanking both sides, AAV2-HBO with an LPQTG tag [SEQ ID NO: 43] located at position 587 with 3 GGSGS linkers flanking both sides, AAV9-A with an LPQTG tag [SEQ ID NO: 43] located at position 455 with 3 GGSGS [SEQ ID NO: 47] linkers flanking both sides and AAV9-A with an LPQTG tag [SEQ ID NO: 43] located at position 589 with 3 GGSGS [SEQ ID NO: 47] linkers flanking both sides. Arrow: conjugated product.

FIG. 10: GGG-Biotin. AAV2-HBO with an LPQTG tag [SEQ ID NO: 43] located at position 587 with 1 GGSGS [SEQ ID NO: 47] linker flanking both sides.

FIG. 11: HER2 vs GFP. AAV2-HBO with an LPQTG tag [SEQ ID NO: 43] located at position 453 with 3 GGSGS [SEQ ID NO: 47] linkers flanking both sides, AAV9-A with an LPQTG tag [SEQ ID NO: 43] located at position 589 with 3 GGSGS [SEQ ID NO: 47] linkers flanking both sides. Arrow: conjugated product.

FIG. 12: LPQTG vs LPETG in AAV2_VR-VIII and AAV9_VR-IV. AAV2-HBO with an LPQTG tag [SEQ ID NO: 43] located at position 587 with 3 GGSGS [SEQ ID NO: 47] linkers flanking both sides, AAV2-HBO with an LPETG tag [SEQ ID NO: 44] located at position 587 with 3 GGSGS [SEQ ID NO: 47] linkers flanking both sides, AAV9-A with an LPQTG tag [SEQ ID NO: 43] located at position 455 with 3 GGSGS [SEQ ID NO: 47] linkers flanking both sides and AAV9-A with an LPETG tag [SEQ ID NO: 44] located at position 455 with 3 GGSGS [SEQ ID NO: 43] linkers flanking both sides. Arrow: conjugated product.

FIG. 13: In vitro targeting. AAV2-HBO with an LPQTG tag [SEQ ID NO: 43] located at position 587 with 3 GGSGS [SEQ ID NO: 47] linkers flanking both sides. (A) % transduced cells, (B) % transduced cells (zoomed in from (A)), (C) flow cytometry results.

FIG. 14: Transduction efficiency comparison for different AAV vectors conjugated with anti-HER2 nanobody relative to non-conjugated vectors. Transduction efficiency is measured by means of GFP fluorescence.

FIG. 15: Comparison of production yields of different constructs. (A) Average viral genome (“Vg”) yield (left Y-axis) and % full viral particles (right Y-axis; black dots) in lysates of producer cell culture. (B) Average viral genome (“Vg”) yield (left Y-axis; black dots) and % full viral particles (right Y-axis) in supernatant of producer cell culture.

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g. any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e. also in the context of other aspects or embodiments of the invention, unless otherwise defined. For example, embodiments directed to products are also applicable to corresponding features of methods and uses.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, alternative combinations of claimed embodiments are encompassed, as would be understood by those in the art.

Unless indicated otherwise, all methods, steps, techniques and manipulations that are not specifically described in detail can be performed and have been performed in a manner known per se, as will be clear to the skilled person. Reference is for example made to standard handbooks as well as to the general background art referred to herein and to the further references cited therein (e.g. Sambrook et al., Molecular cloning: a laboratory manual, ISBN 0879693096, 1989 and the corresponding updated 4^{th} Edition, Cold Spring Harbor Laboratory Press, 2012).

Generally, amino acid sequences and the numbering thereof in the present invention is with reference to the AAV2 VP1 capsid sequence depicted in SEQ ID NO: 7. The same numbering is maintained for the shorter VP2 and VP3 proteins. It is further noted that the insertion of the sortase recognisition sequence, the optional insertion of linker sequences and the deletion of amino acids in the capsid protein backbone leads to polypeptides with a different length. Also for these polypeptide amino acids in the capsid protein backbone remain defined by SEQ ID NO: 7. Sequence similarity between AAV2 capsid proteins and those of other AAV serotypes allow to identify in a sequence alignment the corresponding amino acids with reference to SEQ ID NO: 7. Illustratively, references to corresponding AAV9 positions are also made throughout the present description.

The present invention can be equally applied on AAV capsid sequences which differ from SEQ ID: 7, 8 or 9, such as naturally occurring serotype variants and artificial modifications to alter tropism. Such variant can occur also within the VR regions as is eg the case for the R585A, R588A mutant. Therefore, illustratively AAV2 positions disclosed herein are also intended to cover corresponding amino acid positions in other AAV serotype variants (naturally occurring and artificially generated).

The term “protein” as used throughout this specification generally encompasses macromolecules comprising one or more polypeptide chains, i.e. polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, guanidinylation, phosphorylation, sulfonation, methylation, ubiquitination, signal peptide removal, N-terminal Met removal, etc. The term further also includes protein variants or mutants which carry amino acid sequence variations vis-b-vis a corresponding native proteins, such as, e.g. amino acid deletions, additions and/or substitutions. The term contemplates both full-length proteins and protein parts or fragments, e.g. naturally-occurring protein parts that ensue from processing of such full-length proteins.

The term “polypeptide” as used throughout this specification generally encompasses polymeric chains of amino acid residues linked by peptide bonds. Hence, especially when a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term contemplates both full-length polypeptides and polypeptide parts or fragments, e.g. naturally-occurring polypeptide parts that ensue from processing of such full-length polypeptides.

The term “peptide” as used throughout this specification preferably refers to a short chain of amino acid residues linked by peptide bonds comprising 50 amino acids or less, e.g. 45 amino acids or less, preferably 40 amino acids or less, e.g. 35 amino acids or less, more preferably 30 amino acids or less, e.g. 25 or less, 20 or less, 15 or less or 10 or less amino acids. No strict maximal length is attributed to a peptide to still be considered a peptide. The term peptide may encompass naturally, recombinantly, semi-synthetically or synthetically produced peptides such as discussed for polypeptides above.

The term “amino acid” encompasses naturally occurring amino acids, naturally encoded amino acids or proteinogenic amino acids, non-naturally encoded amino acids, non-naturally occurring amino acids, amino acid analogues and amino acid mimetics that function in a manner similar to the naturally occurring amino acids, all in their D- and L-stereoisomers, provided their structure allows such stereoisomeric forms. Amino acids are referred to herein by either their name, their commonly known three letter codes or by the one-letter codes recommended by the IUPAC-IUB Biochemical Nomenclature Commission. A “naturally encoded amino acid” refers to an amino acid that is one of the 20 common amino acids or pyrrolysine, pyrroline-carboxy-lysine or selenocysteine. The 20 common amino acids are: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr). Also included are amino acid analogues, in which one or more individual atoms have been replaced either with a different atom, an isotope of the same atom, or with a different functional group.

“Encoding” is to be interpreted according to the common interpretation in the art and therefore indicates that a nucleic acid sequence or part(s) thereof corresponds, by virtue of the genetic code of an organism in question to a particular amino acid sequence, e.g. the amino acid sequence of one or more desired proteins or polypeptides, or to another nucleic acid sequence in a template-transcription product (e.g. RNA or RNA analogue) relationship. While numerous references are made throughout the present description that refer to modifications in amino acid positions and/or amino acid sequences, it is evident that upon said modifications are to be introduced into the encoding nucleic acid sequence in embodiments wherein the AAV capsid protein is to be translated from a nucleic acid sequence.

The term “nucleic acid” as used throughout this specification typically refers to a polymer (preferably a linear polymer) of any length composed essentially of nucleoside units. A nucleoside unit commonly includes a heterocyclic base and a sugar group. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g. xanthine, inosine, hypoxanthine) as well as chemically or biochemically modified (e.g. methylated), non-natural or derivatized bases. A nucleic acid can be double-stranded, partly double stranded, or single-stranded. Where single-stranded, the nucleic acid can be the sense strand or the antisense strand. In addition, nucleic acid can be circular or linear.

It is appreciated that a skilled person is capable of assessing sequence Identity between sequences. Since methods and tools to verify sequence identity between different sequences of amino acids or nucleic acids are well known. Such tools include (Protein) BLAST, ClustalW2, SIM alignment tool, TranslatorX, and T-COFFEE. The percentage of identity between two sequences may show minor differences depending on the algorithm choice and parameters. The term “sequence identity” as used herein refers to the relationship between sequences at the nucleotide (or amino acid) level. The expression “% identical” is determined by comparing optimally aligned sequences, e.g. two or more, over a comparison window wherein the portion of the sequence in the comparison window may comprise insertions and/or deletions as compared to the reference sequence for optimal alignment of the sequences. The reference sequence does not comprise insertions or deletions. A reference window is chosen and the “% identity” is then calculated by determining the number of nucleotides (or amino acids) that are identical between the sequences in the window, dividing the number of identical nucleotides (or amino acids) by the number of nucleotides (or amino acids) in the window and multiplying by 100. Unless indicated otherwise, the sequence identity is calculated over the whole length of the reference sequence. An example procedure to determine the percent identity between a particular amino acid sequence and the amino acid sequence of a query polypeptide will entail aligning the two amino acid sequences using the Blast 2 sequences (Bl2seq) algorithm, available as a web application or as a standalone executable programme (BLAST version 2.2.31+) at the NCBI web site (www.ncbi.nlm.nih.gov), using suitable algorithm parameters. An example of suitable algorithm parameters include: matrix=Blosum62, cost to open a gap=11, cost to extend a gap=1, expectation value=10.0, word size=3). A skilled person readily appreciates that any sequences represented in sequence databases or in the present specification may be of precursors of peptides, polypeptides, proteins, or nucleic acids and may include parts which are processed away from mature molecules.

“Adeno-associated virus”, commonly abbreviated as “AAV” refers to a nonpathogenic parvovirus composed of a 4.7 kb single-stranded DNA genome within a non-enveloped, icosahedral capsid. Both the full length term and the abbreviation thereof may be used to refer to the virus itself or derivatives thereof. The AAV genome contains three AAV promoters (i.e. p5, p19, and p40; names referring to their relative map locations) which are responsible for the expression of two open reading frames encoding the rep and cap genes. The two rep promoters (p5 and p19), coupled with the differential splicing of the single AAV intron, drive the production of four Rep proteins (Rep 78, Rep 68, Rep 52, and Rep 40) from the rep gene. The distinct Rep proteins have different enzymatic properties that are involved in various aspects of viral replication. The cap gene is expressed from the p40 promoter and it encodes the three capsid proteins Viral Protein 1 (VP1), Viral Protein 2 (VP2), and Viral Protein 3 (VP3) by alternative splicing and non-consensus translational start sites. VP1, VP2, and VP3 are involved in the encapsidation of AAV (i.e. VP1, VP2, and VP3 are AAV capsid proteins). VP1, VP2, and VP3 therefore have overlapping sequences with VP3 being contained entirely within the sequence of VP2, which is, in turn, contained within VP1. The regions with the highest structural variation (VR) have been annotated in the art as VR-I to VR-IX. The present invention is directed to sequence manipulation within the VR-I, VR-IV, and VR-VIII regions. In addition, the invention encompasses sequence manipulation within the VP1-VP2 transition region (i.e. the region marking the end of the unique N-terminal VP1 portion unique for each of the VP proteins and the N-terminus of the VP2 protein sequence). A single consensus polyadenylation site is located at map position 95 of the AAV genome. Additionally, an open reading frame which is present as an alternate reading frame within the cap gene produces the assembly-activating protein (AAP), a viral protein that localizes AAV capsid proteins to the nucleolus and functions in the capsid assembly process. The AAV genome comprises inverted terminal repeats at both ends.

It is to be appreciated that the term “AAV” as used herein covers all subtypes and/or serotypes (naturally occurring and recombinant forms; rAAV), except where required or explicitly indicated otherwise. The term “AAV” therefore encompasses AAV1, AAV2, AAV3, AAV4, AAV5, AAV6, AAV7, AAV8, AAV9, AAV10, AAV11, AAV12, AAV13, avian AAV, bovine AAV, canine AAV, equine AAV, primate AAV, non-primate AAV, and ovine AAV, or any combination thereof. A skilled person understands that the above indications refer to the subjects that can be infected by said AAV. For example, primate AAV refers to AAV that is able to infect primates. The genomic sequences of various AAV serotypes, their native inverted terminal repeats (ITRs), Rep proteins, and capsid subunits have been described in the art. Such sequences may be found in the literature or in public repositories such as GenBank, UniProt, etc. The term “AAV” equally encompasses mosaic AAVs and chimeric AAVs that contain custom-designed AAV capsid protein that do not naturally occur in nature (including AAVs obtained through e.g. directed evolution). It has been described in the art that the genomic diversity among different serotypes is most concentrated on hypervariable regions (HVRs) of the virus capsid, which influences the tissue tropism of the AAV. It is known to a skilled person that the tissue tropisms of AAV vectors are dependent on other elements (i.e. parameters) such as cell surface receptors, cellular uptake, intracellular processing of the AAV, nuclear delivery of the AAV genome, uncoating of the AAV, and second-strand DNA conversion. Preferred AAV serotypes in the context of the present invention are AAV2 and AAV9, but it is envisaged that any embodiment described herein is equally applicable to any AAV serotype, including mosaic AAVs and chimeric AAVs.

The term “tropism” as used herein refers to the preferred targeting of specific host species or specific cell types within a host species by a virus (in the present context by AAV). For certain viruses, the tropism of a virus describes the virus's relative preferences. A virus can be considered to have a similar (or identical) tropism when compared to another virus if the viruses prefer the same characteristics (e.g. the second virus is also more successful in infecting the same cells (i.e. the same cell type), even if the absolute transduction efficiencies are not similar. It may be that a second virus might be more efficient than a first virus at infecting every given cell type tested, but if the relative preferences are similar (or identical), the second virus is generally considered in the art to have a similar (or identical) tropism as the first virus.

An “AAV (viral) particle”, “AAV virion” or “AAV (vector) particle” refers to a viral particle composed of at least one AAV capsid protein and an encapsidated single-strand nucleic acid strand. In certain embodiments, the AAV particle comprises a heterologous nucleic acid sequence (i.e. a nucleic acid other than a wild-type AAV genome such as a transgene to be delivered to a eukaryotic cell, such as but not limited to a mammalian cell or insect cell). Generally, the related term “AAV capsid” refers to the outer surface (i.e. the capsid) of the AAV particle. The AAV capsid contains 60 copies (in total) of the three VPs, predicted to be present in the capsid in a VP1:VP2:VP3 ratio of 1:1:10. Hence, references throughout the present specification to “AAV capsid protein” encompasses each of the VP1, VP2, and VP3 proteins, or if indicated any selection thereof. It is to be appreciated that in instances throughout the present specification wherein the term “conjugated AAV particle” is used, this indicates an AAV particle that has been subjected to a sortase conjugation reaction.

“Sortase”, interchangeably used with “sortase enzyme” refers to a family of enzymes that, in nature, play a role in the formation of the bacterial cell wall by covalently linking specific surface proteins to a peptidoglycan. In the broadest interpretation, sortases can be defined as enzymes that recognize a stretch of amino acids, cleave site-specifically within that stretch of amino acids, and finally attach a substrate moiety immediately C-terminal from the cleavage site through a peptide bond with the N-terminal residue of an acceptor moiety. Alternatively worded, sortase enzymes recognize a sortase recognition motif in a substrate protein and carry out a transpeptidation reaction. In a first reaction step, the sortase cleaves a peptide bond in the sortase recognition motif, forming an acyl intermediate with the cleaved sortase recognition motif. In a second reaction step, the sortase binds to an acceptor moiety bearing a sortase acceptor motif (typically at least one Glycine or a stretch of Glycines) and transfers the acyl intermediate. The reaction results in the formation of a new peptide bond between the substrate protein and the acceptor moiety. Alternative terms interchangeably used herein and in the art to refer to a “sortase recognition motif” include “sortase recognition sequence” and “sortase recognition tag”.

Different sortases have been described in the art. Non-limiting examples hereof include sortase enzymes naturally occurring in gram-positive enzymes. Preferred sortase enzymes include sortase A, sortase B, sortase C, sortase D, sortase E, and sortase F. More specific sortase subfamilies have been described, which are also encompassed by the term “sortase”. It is well within the capacities of a person skilled in the art to assign an identified sortase to the correct class and/or subfamily based on its sequence or functional characteristics (e.g. transpeptidation activity). When a general reference throughout the present description is made to “sortase”, it is evident that all sortase types and subtypes, naturally occurring and artificially engineered are envisaged, unless specifically indicated otherwise.

The AAV capsid proteins as described herein aim to accommodate physical linkage between the AAV capsid protein and a heterologous conjugate (molecule). The term “heterologous”, interchangeably used in the art with the term “exogenous” indicates that a certain moiety (in the context of the present invention the conjugate molecule) is not occurring in the natural, unmodified version of the AAV. Thus, in accordance with the generally accepted nomenclature in the fields of molecular biology and biotechnology, “heterologous conjugate” and “heterologous conjugate molecule” refer to a molecule that is present in a host organism which does not naturally contains or expresses said molecule.

The inventors have unexpectedly found that sortase recognition sequences can be introduced at specific internal locations in capsid sequences of AAV particles to enable the conjugation of targeting molecules with the AAV capsids (to produce conjugated AAV particles) to more efficiently and specifically target tissues and cells, whilst maintaining similar production efficiencies when compared to the appropriate parental AAV serotype. This finding is remarkable for a number of reasons:
For biotechnological applications, there is a general teaching in the art that sortase recognition sequences need to be located at the C-terminus of a protein, or at least in the near proximity thereof to be functional. The notion that internally located sortase recognition sequences in AAV capsid proteins would be both functional and accessible is unexpected.
Notably, even after conducting the sortase reaction, structural and functional integrity of the (now split) capsid proteins forming the (conjugated) AAV capsid is maintained. Even upon the introduction of extended linker sequences, the AAV capsid proteins remained susceptible to being engaged by the sortase enzyme. Moreover it was surprisingly found that insertion of a sortase recognition sequence in combination with relatively long linker sequences (even exceeding a length of 34 amino acids indicated in the art as the maximum insertion length) in surface-exposed regions does not negatively affect the structural nor the functional properties of the viral capsid protein.
It could not be envisaged that the VP1-VP2 transition region, the VR-I region, the VR-IV region, and the VR-VIII region are accessible to such an extent that both a sortase and an acceptor moiety can be brought into a close proximity for completing the sortase reaction.
Unexpectedly, the findings by the inventors can be readily reproduced in (or extrapolated to) to different AAV serotypes. The sequence diversity of the AAV capsid proteins among serotypes does not form a barrier to insert and use an internal sortase recognition sequence.

Accordingly, the invention provides adeno-assisted virus (AAV) capsid proteins characterized in that a sortase recognition sequence is inserted within their protein sequence, particularly to VP1, VP2, or VP3 capsid proteins. More particularly the invention relates to an adeno-assisted virus (AAV) capsid protein characterized in that in one or more of the VP1-VP2 transition region, VR-I region, VR-IV region and VR-VIII region a sortase recognition sequence is inserted. Optionally, the AAV capsid protein is VP1 and a sortase recognition sequence is inserted in VP1-VP2, VR-I, VR-IV, VR-VIII, or any combination thereof. Preferably, the AAV capsid protein is VP1 and a sortase recognition sequence is inserted in VR-I, VR-IV, VR-VIII, or any combination thereof. Alternatively, the AAV capsid protein is VP2 and a sortase recognition sequence is inserted in VR-I, VR-IV, VR-VIII, or any combination thereof. Alternatively, the AAV capsid protein is VP3 and a sortase recognition sequence is inserted in VR-I, VR-IV, VR-VIII, or any combination thereof. Given that the sequence of VP3 is fully encompassed by the sequence of VP2, which is in turn fully encompassed by the sequence of VP1, the AAV capsid protein may be a protein comprising the sequence of a VP3 protein defined herein. Alternatively or additionally, the AAV capsid protein may be a protein comprising the sequence of a VP2 protein defined herein. Yet alternatively or additionally, the AAV capsid protein may be a protein comprising the sequence of a VP3 protein described herein. It is evident that in the envisaged aspect, the VP1, VP2, or VP3 sequence differs from the canonical sequence of these proteins due to the presence of the one or more sortase recognition motifs. Hence, the AAV capsid proteins subject of the invention do not share 100% sequence identity with naturally occurring VP1, VP2, or VP3 proteins or their encoding sequences. Therefore, in certain embodiments the AAV capsid protein comprises an amino acid sequence which is at least at least about 80% identical, preferably at least about 85% identical, at least about 90% identical, at least about 95% identical, or at least about 98% identical to the amino acid sequence of a naturally occurring (i.e. wild-type) AAV capsid protein, wherein the capsid protein comprises at least one sortase recognition sequence. In preferred embodiments, the AAV capsid protein comprises an amino acid sequence which is at least at least about 80% identical, preferably at least about 85% identical, at least about 90% identical, at least about 95% identical, or at least about 98% identical to the amino acid sequence of a naturally occurring (i.e. wild-type) AAV2 or AAV9, preferably AAV2 capsid protein.

In preferred embodiments, the sortase recognition sequence is operably linked to an N-terminal linker (i.e. a linker sequence N-terminal of the sortase recognition motif).

In alternative preferred embodiments, the sortase recognition sequence is operably linked to an C-terminal linker (i.e. a linker sequence C-terminal of the sortase recognition motif). In more preferred embodiments, the sortase recognition sequence is flanked by an N-terminal and a C-terminal linker sequence. Optionally, the linker sequence is a GGGGS [SEQ ID NO: 39] sequence or any plurality thereof, or a GGSGS [SEQ ID NO: 47] sequence or any plurality thereof. Optionally, the linker sequence comprises a GGGGS [SEQ ID NO: 39] sequence, comprises a GGSGS [SEQ ID NO: 47] sequence, or comprises any combination thereof.

It is evident that for any sortase recognition sequence described herein and optional linker sequences each of the sequences have to be operably linked to the N-terminal and C-terminal portion of the AAV capsid protein. The term “operably linked” is well known to a person of ordinary skill in the molecular biology and refers to an arrangement of elements wherein the components so described are configured so as to perform their usual function. Thus, sortase recognition sequences operably linked to an internal position within an AAV capsid protein sequence will not interrupt translation of said AAV capsid protein. The control sequences need not be contiguous with the coding sequence, so long as they function to direct the expression thereof.

Optionally, the sortase recognition sequence is a sequence corresponding to a sortase recognition sequence of a naturally occurring sortase enzyme, such as but not limited to a naturally occurring sortase enzyme in gram-positive bacteria. Preferably, the sortase recognition sequence is a sequence corresponding to a general sortase recognition sequence of a sortase selected from the group consisting of: sortase A, sortase B, sortase C, sortase D, sortase E, and sortase F. Preferably, the sortase recognition sequence is a sequence corresponding to the general sortase recognition sequence of sortase A.

In certain embodiments, the AAV capsid protein comprises two or more sortase recognition sequences of a sortase selected from the group consisting of: sortase A, sortase B, sortase C, sortase D, sortase E, and sortase F. It is preferred that in embodiments wherein multiple sortase recognition sequences are inserted, said sortase recognition sequences are inserted at distinct locations of the AAV capsid protein. The sortase recognition sequences in such embodiments may be identical sortase recognition sequences or any combination of sortase recognition sequences of a sortase selected from the group consisting of: sortase A, sortase B, sortase C, sortase D, sortase E, and sortase F. Preferably, the AAV capsid protein comprises at least a sortase A recognition sequence.

Optionally, the sortase recognition sequence is a sequence which has a general sequence motif selected from the group consisting of: LPXTG [SEQ ID NO: 10], NPXTG [SEQ ID NO:40], LPXTA [SEQ ID NO: 41], LAXTG [SEQ ID NO: 42], LPXAG [SEQ ID NO: 48], LPXLG [SEQ ID NO: 49], APXTG [SEQ ID NO: 50], LPXSG [SEQ ID NO: 51], FPXTG [SEQ ID NO: 52], XPKTG, [SEQ ID NO: 53], and LPEXG, [SEQ ID NO: 54], wherein X is any amino acid. Preferably, the sortase recognition sequence is a sequence having the general sequence motif LPXTG [SEQ ID NO: 10] wherein X is any amino acid and wherein X is defined independently for Xn and Xm. In yet further preferred embodiments, the sortase recognition sequence is LPQTG [SEQ ID NO: 43], LPETG [SEQ ID NO: 44], LPNTG [SEQ ID NO: 45], LPDTG [SEQ ID NO: 46], NPQTN [SEQ ID NO: 55], QVPTG [SEQ ID NO: 56], LPNTA [SEQ ID NO: 57], LPLTG [SEQ ID NO: 58], APKTG [SEQ ID NO: 59], DPKTG [SEQ ID NO: 60], SPKTG [SEQ ID NO: 61], APATG [SEQ ID NO: 62], LAETG [SEQ ID NO: 63], LPEAG [SEQ ID NO: 64], LPECG [SEQ ID NO: 65], LPESG [SEQ ID NO: 66], or LMVGG [SEQ ID NO: 67]. In most preferred embodiments, the sortase recognition sequence is LPQTG [SEQ ID NO: 43] or LPETG [SEQ ID NO: 44]. An alternative suitable manner of describing the sortase recognition sequence as described herein is by means of the format “Xn-sortase recognition sequence-Xm”. This format therefore indicates the optional presence of a linker sequence wherein “X” indicates any amino acid and “n” and “m” are integers indicating the length of the linker sequence (i.e. the amount of amino acids). Evidently, X can be defined for Xn and Xm independently of one another. Hence, in certain embodiments, the sortase recognition sequence is immediately preceded by the sequence Xn, and immediately followed by the sequence Xm. Thus, the sortase recognition sequences described herein may alternatively be indicated by: Xn-LPXTG-Xm [SEQ ID NO: 10], Xn-NPXTG-Xm [SEQ ID NO: 40], Xn-LPXTA-Xm [SEQ ID NO: 40], Xn-LAXTG-Xm [SEQ ID NO: 42], Xn-LPXAG-Xm [SEQ ID NO: 48], Xn-LPXLG-Xm [SEQ ID NO: 49], Xn-APXTG-Xm [SEQ ID NO: 50], Xn-LPXSG-Xm [SEQ ID NO: 51], Xn-FPXTG-Xm [SEQ ID NO: 52], Xn-XPKTG-Xm, [SEQ ID NO: 53], and Xn-LPEXG-Xm, [SEQ ID NO: 54], wherein X is any amino acid, and n and m range from 0 to 25. Preferably, the sortase recognition sequence is a sequence having the general sequence motif Xn-LPXTG-Xm [SEQ ID NO: 10] wherein X is any amino acid and n and m range from 0 to 25. More preferably, the sortase recognition sequence is Xn-LPQTG-Xm [SEQ ID NO: 43], Xn-LPETG-Xm [SEQ ID NO: 44], Xn-LPNTG-Xm [SEQ ID NO: 45], Xn-LPDTG-Xm [SEQ ID NO: 46], Xn-NPQTN-Xm [SEQ ID NO: 55], Xn-QVPTG-Xm [SEQ ID NO: 56], Xn-LPNTA-Xm [SEQ ID NO: 57], Xn-LPLTG-Xm [SEQ ID NO: 58], Xn-APKTG-Xm [SEQ ID NO: 59], Xn-DPKTG-Xm [SEQ ID NO: 60], Xn-SPKTG-Xm [SEQ ID NO: 61], Xn-APATG-Xm [SEQ ID NO: 62], Xn-LAETG-Xm [SEQ ID NO: 63], Xn-LPEAG-Xm [SEQ ID NO: 64], Xn-LPECG-Xm [SEQ ID NO: 65], Xn-LPESG-Xm [SEQ ID NO: 66], or Xn-LMVGG-Xm [SEQ ID NO: 67]. Most preferably, the sortase recognition sequence is Xn-LPQTG-Xm [SEQ ID NO: 43] or Xn-LPETG-Xm [SEQ ID NO: 44].

As described above, in certain embodiments the sortase recognition sequence is preceded and/or followed by a linker sequence (i.e. embodiments wherein the sortase recognition sequence is preceded or following by an Xn and/or an Xm sequence wherein n and/or m is at least 1 and wherein X for Xn and Xm can be defined independently of one another). Hence, the invention envisages embodiments wherein n and/or m are 0, but equally envisages embodiments wherein n and/or m is at least 1. Therefore, in certain embodiments n and/or m is an integer from 0 to 25, such as at least 2, at least 3, at least 4 or at least 5, preferably from 5 to 20, such as from 10 to 15.

Optionally, n and/or m is an integer from 15 to 20, or between 15 and 20. Optionally, n and/or m is an integer from 10 to 15, or between 10 and 15. Optionally, n and/or m is an integer from 5 to 10, or between 0 to 5. Optionally, n and/or m is 25, 24, 23, 22, 21, 20, 19, 18, 17, 16, 15, 14, 13, 12, 11, 10, 9, 8, 7, 6, 5, 4, 3, 2, 1, or 0. Further envisaged are embodiments wherein n is 0 and m is an integer from 0 to 25, preferably 0 to 20. Equally envisaged are embodiments wherein m is 0 and n is an integer from 0 to 25, preferably 0 to 20.

The exact sequences of the linker sequences Xn and Xm are not particularly limiting for the invention. However, preferred Xn and Xm sequences (or characteristics thereof) are described in the following paragraphs. Preferably, the linker sequence Xn, Xm, or both Xn and Xm consist of at least 65%, preferably at least 75%, preferably at least 80%, preferably at least 85%, preferably at least 85%, more preferably at least 90%, more preferably at least 95% of Glycine, Serine, Threonine, and Alanine. More preferably, the linker sequence Xn, Xm, or both Xn and Xm consist of Glycine, Serine, Threonine, and Alanine. Alternatively, the linker sequence Xn, Xm, or both Xn and Xm consist of at least 65%, preferably at least 75%, preferably at least 80%, preferably at least 85%, preferably at least 85%, more preferably at least 90%, more preferably at least 95% of Glycine and Serine. In further alternative embodiments, the linker sequence Xn, Xm, or both Xn and Xm consist of Glycine and Serine. In particular embodiments the linker sequence is GGSGS [SEQ ID NO: 47].

Each of the regions described herein wherein a sortase recognition sequence may be inserted have been described at numerous occasions throughout the art and it is therefore well within the capacities of a person having ordinary skill in the art to identify these regions. Optionally, the VP1-VP2 transition region is defined by SEQ ID NO: 1 [PVKTAP]. Optionally, the VR-I region is defined by SEQ ID NO: 2 [SSQSGASN]. Optionally, the VR-IV region is defined by SEQ ID NO: 3 [SRTNTPSGTTTQSRLQFSQAGASDIRDQS]. Optionally, the VR-VIII region is defined by SEQ ID NO: 4 [QYGSVSTNLQRGNRQAATADVNTQGV]. In preferred embodiments, the VR-I region is defined by SEQ ID NO: 2 [SSQSGASN], the VR-IV region is defined by SEQ ID NO: 3 [SRTNTPSGTTTQSRLQFSQAGASDIRDQS], and the VR-VIII region is defined by SEQ ID NO: 4 [QYGSVSTNLQRGNRQAATADVNTQGV]. In a further preferred embodiment, the VP1-VP2 transition region is defined by SEQ ID NO: 1 [PVKTAP], the VR-I region is defined by SEQ ID NO: 2 [SSQSGASN], the VR-IV region is defined by SEQ ID NO: 3 [SRTNTPSGTTTQSRLQFSQAGASDIRDQS], and the VR-VIII region is defined by SEQ ID NO: 4 [QYGSVSTNLQRGNRQAATADVNTQGV]. It is to be understood that SEQ ID NO: 1 to SEQ ID NO: 4 correspond to sequences wherein the sortase recognition motif is not inserted, i.e. sequences which the inventors have found to be receptable to one or more sortase recognition motifs optionally flanked by one or more linkers. Equally envisaged are corresponding amino acid sequences in AAV serotypes that are not AAV2.

In embodiments wherein the sortase recognition sequence is inserted in the VR-IV region, the sortase recognition sequence may preferably be inserted in the fragment with SEQ ID NO: 5 [TPSGTTTQS] and/or the insertion in the VR-VIII region is in the fragment with SEQ ID NO: 6 [LQRGNRQAA]. In such embodiments, the sortase recognition sequence may be inserted after the 1^{st}, 2^{nd}, 3^{rd}, 4^{th}, 5^{th}, 6^{th}, 7^{th}, 8^{th}, or 9^{th} amino acid of SEQ ID NO: 5. In alternative such embodiments, the sortase recognition sequence may be inserted after the 1^{st}, 2^{nd}, 3^{rd}, 4^{th}, 5^{th}, 6^{th}, 7^{th}, 8^{th}, or 9^{th} amino acid of SEQ ID NO: 6. In yet alternative embodiments, a first sortase recognition sequence may be inserted after the 1^{st}, 2^{nd}, 3^{rd}, 4^{th}, 5^{th}, 6^{th}, 7^{th}, 8^{th}, or 9^{th} amino acid of SEQ ID NO: 5 and a second sortase recognition sequence may be inserted after the 1^{st}, 2^{nd}, 3^{rd}, 4^{th}, 5^{th}, 6^{th}, 7^{th}, 8^{th}, or 9^{th} amino acid of SEQ ID NO: 6.

The sortase recognition sequences described herein may be inserted at the VP1-VP2 transition region, the VR-I region, the VR-IV region, and/or the VR-VIII region by insertion of the sortase recognition sequence(s) in the genomic sequence of the region(s). The insertion may be an insertion without removal of canonical amino acids in said region(s). In such embodiments, the sortase recognition sequence is introduced in addition to the original sequence. Alternatively, the sortase recognition sequences described herein may be inserted at the VP1-VP2 transition region, the VR-I region, the VR-IV region, and/or the VR-VIII region by insertion and deletion (resulting in a substitution or replacement) in the genomic sequence of the region(s). The deletion or substitution may correspond to any number of amino acids of the VP1-VP2 transition region, the VR-I region, the VR-IV region, and/or the VR-VIII region as long as the structural integrity of said region (and consequently the AAV capsid protein) is maintained. In some embodiments, the functional integrity of the AAV protein can be affected, in that the insertion site interferes with specific functionalities (such as the HSPG binding site of VR-VIII in AAV2), though these will not be critical to the further use of the conjugated protein. However, in further embodiments, both the structural and functional integrity of the region (and consequently the AAV capsid protein) is maintained.

Without withing to be bound by theory, the inventors note that surface-exposed Lysines may decrease the reaction efficiency of sortase-mediated transpeptidation, optionally by allowing generation of unallowed side products. Hence, in embodiments of the invention one or multiple surface-exposed lysine are mutated, such as in glycine, serine or alanine. The choice of which Lysines to target is based on their distance from the loop wherein the insertion site, such as the LPXTG tag is being added.

A skilled person is capable of identification of corresponding amino acids to those disclosed herein for AAV serotypes that are not AAV2. Generally, envisaged are Lysine mutations that are in a close proximity, to the region(s) wherein the sortase recognition sequence is inserted.

Illustratively, Lysines in 30 angstrom vicinity of the VR-I region of AAV2 correspond to K258, K321, K490, K507, K527, K532, K544, K549, K556, K620, K640, K649, K665, K692, and K706; Lysines in 30 angstrom vicinity of the VR-IV region of AAV2 correspond to K258, K309, K313, K321, K490, K507, K527, K532, K544, K549, K556, K620, K640, K649, K665, K688, K692, and K706; and Lysines in 30 angstrom vicinity of the VR-VIII region of AAV2 correspond to K309, K490, K507, K527, K532, K544, K549, K556, K620, K640, K688, and K706.

Illustratively, Lysines in 15 angstrom vicinity of the VR-I region of AAV2 correspond to K258, K507, K527, K549, and K706; Lysines in 15 angstrom vicinity of the VR-IV region of AAV2 correspond to K258, K490, K507, K532, K544, K549, K556, and K665; and Lysines in 15 angstrom vicinity of the VR-VIII region correspond to K490, K507, K527, and K532.

Illustratively, a Lysine in 5 angstrom vicinity of the VR-IV region of AAV2 is K549; and a Lysine in 5 angstrom vicinity of the VR-VIII region of AAV2 is K507.

Optionally, the sortase recognition sequence is inserted in the VR-I region and one or more of Lysines at positions K258, K321, K490, K507, K527, K532, K544, K549, K556, K620, K640, K649, K665, K692, K706 in AAV2 or corresponding amino acids in another serotype are mutated. In preferred embodiments, the sortase recognition sequence is inserted in the VR-I region and one or more of Lysines at positions K258, K507, K527, K549 and K706 in AAV2 or corresponding amino acids in another serotype are mutated. Optionally, a sortase A recognition sequence is inserted in the VR-I region and one or more of Lysines at positions K258, K321, K490, K507, K527, K532, K544, K549, K556, K620, K640, K649, K665, K692, K706 in AAV2 or corresponding amino acids in another serotype are mutated. Optionally, the sortase A recognition sequence is Xn-LPXTG-Xm [SEQ ID NO: 10].

Optionally, in the embodiments envisaged herein the sortase recognition sequence is inserted in the VR-IV region and one or more of Lysines at position K258, K309, K313, K321, K490, K507, K527, K532, K544, K549, K556, K620, K640, K649, K665, K688, K692 and K706 in AAV2 or corresponding amino acids in another serotype are mutated. In preferred embodiments, the sortase recognition sequence is inserted in the VR-IV and one or more of Lysines at position K258, K490, K507, K532, K544, K549, K556 and K665 in AAV2 or corresponding amino acids in another serotype are mutated. Optionally, a sortase A recognition sequence is inserted in the VR-IV region and one or more of Lysines at positions K258, K309, K313, K321, K490, K507, K527, K532, K544, K549, K556, K620, K640, K649, K665, K688, K692 and K706 in AAV2 or corresponding amino acids in another serotype are mutated. Optionally, the sortase A recognition sequence is Xn-LPXTG-Xm [SEQ ID NO: 10]. In further preferred embodiments, the sortase recognition sequence LPXTG [SEQ ID NO: 10] is inserted in the VR-IV region and the Lysine at position K549 in AAV2 or a corresponding amino acid in another serotype is mutated, preferably into Glycine or Alanine.

Optionally, the sortase recognition sequence is inserted in the VR-VIII region and one or more of Lysines at position K309, K490, K507, K527, K532, K544, K549, K556, K620, K640, K688 and K706 in AAV2 or corresponding amino acids in another serotype are mutated. In preferred embodiments, the sortase recognition sequence is inserted in the VR-VIII region and one or more Lysines at position K490, K507, K527 and K532 in AAV2 or corresponding amino acids in another serotype are mutated. Optionally, a sortase A recognition sequence is inserted in the VR-VIII region and one or more of Lysines at position K309, K490, K507, K527, K532, K544, K549, K556, K620, K640, K688 and K706 in AAV2 or corresponding amino acids in another serotype are mutated. Optionally, the sortase A recognition sequence is Xn-LPXTG-Xm [SEQ ID NO: 10].

In further preferred embodiments, the sortase recognition sequence LPXTG [SEQ ID NO: 10] is inserted in the VR-VIII region and the Lysine at position K507 or a corresponding amino acid in another serotype is mutated, preferably into Glycine or Alanine.

A further aspect of the invention is directed to a nucleic acid (i.e. a nucleic acid sequence) encoding any of the AAV capsid proteins described herein and their use in methods for producing AAV particles. Hence, encompassed are nucleic acid sequences that encode any of the AAV capsid proteins described herein that have a sortase recognition sequence in one or more of the VP1-VP2 transition region, VR-I region, VR-IV region and VR-VIII region. The nucleic acid may be DNA, RNA, variants, or any combinations of DNA and RNA. Methods for the construction of nucleic acid constructs of the present disclosure are well known. The nucleic acid can be a nucleic acid further comprising a promotor sequence. The term “promoter” as defined herein is a region of DNA that initiates transcription of a particular gene and hence enables a gene to be transcribed. A promoter is recognized by RNA polymerase, which then initiates transcription. Thus, a promoter contains a DNA sequence that is either bound directly by, or is involved in the recruitment, of RNA polymerase. A promoter sequence can also include “enhancer regions”, which are one or more regions of DNA that can be bound with proteins (namely the trans-acting factors) to enhance transcription levels of genes in a gene-cluster. The enhancer, while typically at the 5′ end of a coding region, can also be separate from a promoter sequence, e.g., can be within an intronic region of a gene or 3′ to the coding region of the gene. Promoters may be located in close proximity of the start codon of genes, in preferred embodiments on the same strand and typically upstream (5′) of the gene. Promoters may vary in size, and are preferably from about 100 to 1000 nucleotides long.

Hence, the term nucleic acid encompasses (recombinant) nucleic acid vectors and (recombinant) nucleic acid expression vectors, which are a further aspect of the invention. Nucleic acid (expression) vectors are known to a skilled person to be suitable to transport the nucleic acid of the invention into a cell within an environment, such as, but not limited to, an organism, tissue, or cell culture. Such vectors are useful for producing, by means of illustration open reading frames encoding AAV capsid proteins subject of the present description. In such embodiments, the AAV capsid proteins subject of the present invention may be expressed by the nucleic acid in in vitro or in vivo conditions (i.e. the nucleic acid encodes at least the amino acid sequence of the AAV capsid protein). Hence, the nucleic acids described herein may be suitable for producing, or suitable for assisting in producing AAV particles and ultimately conjugated AAV particles, said (optionally conjugated) AAV particles being described in detail further throughout the present specification. A recombinant expression vector refers to a nucleic acid encoding a protein, wherein the nucleic acid can express the encoded protein, in the present context an AAV capsid protein. Examples of such vectors include plasmids, nucleic acid viral vectors and viral genomes (including both DNA and RNA genomes). Thus, recombinant AAV vectors are envisaged by the present disclosure.

The term “recombinant AAV vector”, interchangeably used with terms such as “recombinant AAV”, “recombinant AAV virus”, and “recombinant AAV virus particle” indicate that the genome DNA encapsulated in the AAV virus capsid contains a heterologous nucleic acid. In the recombinant AAV vectors of the invention, at least the AAV capsid protein is replaced with a heterologous nucleic acid comprising an AAV capsid protein characterised by one or more sortase recognition sequences in one or more of the the VP1-VP2 transition region, VR-I region, VR-IV region and/or VR-VIII region. The encoded sequences and elements contained by the vector can be expressed in suitable host cells by any means appropriate to introduce the vector into the interior of said cells. Suitable methods include by means of illustration and not limitation infection, transformation, transduction, and transfection. In addition to a therapeutic gene and/or a reporter gene (i.e. the envisaged “payload” to be delivered to target cells), the vector may comprise a plurality of components (i.e. elements, features) having as function the modulation of expression, including but not limited to a promoter sequence, a transcription initiation sequence, an enhancer sequence, an intron, a kozak sequence, a polyA sequence, a selection element, or an origin of replication.

In certain embodiments, the nucleic acid comprises a sequence encoding an AAV capsid protein having an amino acid sequence which is at least at least about 80% identical, preferably at least about 85% identical, at least about 90% identical, at least about 95% identical, or at least about 98% identical to the amino acid sequence of a naturally occurring (i.e. wild-type) AAV capsid protein, wherein the capsid protein comprises at least one sortase recognition sequence. In preferred embodiments, the nucleic acid comprises a sequence encoding an AAV capsid protein having an amino acid sequence which is at least at least about 80% identical, preferably at least about 85% identical, at least about 90% identical, at least about 95% identical, or at least about 98% identical to the amino acid sequence of a naturally occurring (i.e. wild-type) AAV2 or AAV9, preferably AAV2 capsid protein.

The invention aims to provide a robust and efficient means to couple heterologous conjugate molecules to an AAV particle (resulting in a conjugated AAV particle), and more particularly to AAV capsid protein. Therefore, yet a further aspect of the invention is directed to AAV particles comprising a sortase recognition sequence in one or more of the VP1-VP2 transition region, VR-I region, VR-IV region and VR-VIII region of an AAV capsid protein. A skilled person appreciates that after conducting a sortase conjugation reaction the sortase recognition sequence will be modified since the C-terminal Glycine residue residue is “cleaved” and the remaining portion of the sortase recognition sequence is ligated to a distinct Glycine from the conjugate molecule. Hence, a skilled person appreciates that both AAV particles that serve as starting material (i.e. “input” material) for the conjugation are envisaged, but equally AAV particles that are obtained by conducting the sortase conjugation reaction (i.e. the “output” material; conjugated AAV particles). Generally, throughout the present disclosure the sortase recognition sequence part of the AAV capsid protein after the sortase conjugation reaction are indicated as “modified sortase recognition sequence”, or alternatively “remnant sortase recognition sequence”. It is consequently to be appreciated that a remnant sortase recognition sequence as referred to herein physically connects, and preferably operably links the conjugated AAV capsid protein with a heterologous conjugate molecule, such as but not limited to those conjugate molecules described further below.

Alternatively worded, the present invention thus provides in AAV particles comprising a genomically modified AAV capsid protein, wherein the genomic modification is the presence of a sortase recognition sequence in one or more of the VP1-VP2 transition region, VR-I region, VR-IV region and/or VR-VIII region. The genomic modification is therefore to be considered vis-b-vis any AAV particle wherein the AAV capsid protein does not have a sortase recognition sequence in one or more of the VP1-VP2 transition region, VR-I region, VR-IV region and/or VR-VIII region.

Hence, in certain embodiments, the AAV particle comprises an AAV capsid protein characterised by the presence of a sortase recognition sequence in or more of the VP1-VP2 transition region, VR-I region, VR-IV region and/or VR-VIII region. In alternative embodiments, the conjugated AAV particle comprises an AAV capsid protein characterised by the presence of a remnant sortase recognition sequence in or more of the VP1-VP2 transition region, VR-I region, VR-IV region and/or VR-VIII region operably linked to a heterologous conjugate molecule. Given the internal position of the sortase recognition sequence and the particulars of the sortase conjugation reaction, the AAV capsid protein (which may be a VP1, VP2, or VP3 protein) post conjugation reaction will effectively be present in the AAV capsid as two separate proteins; a first N-terminal protein comprising the VP portion N-terminal of the remnant sortase recognition sequence, the remnant sortase recognition sequence, and the conjugate molecule; and a second C-terminal protein comprising the VP portion C-terminal of the (initial) sortase recognition sequence. The sortase recognition sequence is inserted in the AAV capsid protein sequence such that both portions retain structural integrity after conducting the conjugation reaction.

With respect to the conjugated AAV particles, the inventors have further unexpectedly observed that conjugated AAV particles wherein a relatively lower portion of the AAV capsid protein is conjugated demonstrate a more pronounced improvement in transduction efficiencies when compared to conjugated AAV particles wherein a relatively higher portion of the AAV capsid protein is conjugated. It is evident that a lower amount of conjugated AAV capsid proteins in the conjugated AAV particle can be the result of a lower amount of AAV capsid proteins that comprise a sortase recognition sequence or the result of (optionally deliberately) suboptimal parameters for conducting the conjugation reaction. Both embodiments are envisaged by the present disclosure.

Therefore, optionally, the ratio of capsid protein that is not conjugated (i.e. unmodified capsid protein or unconjugated capsid protein) over conjugated capsid protein (i.e. modified capsid protein) in the conjugated AAV particles is between about 1/59 and about 59/1, preferably between about 1/20 and about 20/1, preferably between about 1/15 and about 15/1, preferably between about 1/10 and about 10/1, more preferably between about 1/9 and about 9/1, more preferably between about 1/8 and about 8/1, more preferably between about 1/7 and about 7/1, more preferably between about 1/6 and about 6/1, more preferably between about 1/5 and about 5/1. In preferred embodiments, the ratio of capsid protein that is not conjugated (i.e. unmodified capsid protein) over conjugated capsid protein (i.e. modified capsid protein) in the conjugated AAV particles is less than about 1/5, preferably less than about 1/10, preferably less than about 1/15, preferably less than about 1/20. In preferred embodiments, the conjugated AAV particles comprise at least 1 conjugated capsid protein (i.e. at most 59 nonconjugated capsid proteins). In preferred embodiments, the conjugated AAV particles comprise from 1 to 20 conjugated capsid proteins, preferably from 1 to 15 conjugated capsid proteins, preferably from 1 to 10 conjugated capsid proteins, more preferably from 1 to 5 conjugated capsid proteins. In a particular embodiment, the conjugated AAV particle comprises 1 conjugated capsid protein. Preferably, the conjugated AAV particle comprises at least 5% conjugated AAV capsid proteins. On the other hand, the present inventors have found that where desirable, the present invention allows for efficient conjugation of the capsid protein, where this is of interest. Accordingly, in particular embodiments, the conjugated AAV particles comprise at least 20, preferably at least 30, more preferably at least 40, such as at least 50 conjugated capsid proteins.

It is generally assumed that each AAV particle will comprise at least one VP1 and at least one VP2, theoretically in the ratio VP1:2:3 or 5:5:50. Accordingly, it will be understood that by introduction of the sortase recognition sequence in one or more regions of capsid proteins, the number of conjugated proteins in the particle can be influenced.

Optionally, the VP1, VP2, or VP3 proteins in the (optionally conjugated) AAV particle that comprise a sortase recognition sequence in one or more of the VP1-VP2 transition region, VR-I region, VR-IV region and VR-VIII region have an identical amino acid sequence (i.e. there are no VP1 capsid proteins comprised in a single assembled AAV particle that have sequences that differ from one another, there are no VP2 capsid proteins comprised in a single assembled AAV particle that have sequences that differ from one another, and/or there are no VP3 capsid proteins comprised in a single assembled AAV particle that have sequences that differ from one another). In such embodiments, the sortase recognition sequence may be different, and/or the sortase recognition sequence(s) may be inserted in different regions of the AAV capsid protein.

The particulars of the heterologous conjugate molecule are not particularly limiting for the invention on condition that these are receptable for sortase-mediated conjugation molecules. Generally, this entails the presence of an N-terminal triglycine sequence (i.e. GGG). By means of illustration and not limitation, suitable heterologous conjugate molecules may be small molecules, carbohydrates, lipids, or proteins.

In preferred embodiments, the heterologous conjugate molecule is a targeting moiety. The term “targeting moiety” as used herein encompasses any molecule that is able to bind to a certain tissue, cell type, and/or organ with a preference over other respectively tissues, target and/or organs. The particulars of the targeting moiety are not particularly limiting for the invention and therefore include by means of illustration and not limitation ligands of cell receptors and proteins which bind to cell surface proteins. Preferred heterologous conjugate molecules therefore include antibodies and antibody fragments such as but not limited to nanobodies. Optionally, in embodiments wherein the heterologous conjugate molecule is an antibody or antibody fragment such as an antibody, the heterologous conjugate molecule specifically binds to Human Epidermal growth factor Receptor 2 (HER2).

A related aspect of the invention is directed to the conjugated AAV particles as described herein for use as a medicament. Hence, a method of treatment of a subject in need thereof is encompassed by the invention, said method comprising a step of administration of the conjugated AAV particles to the subject. Also envisaged is the use of conjugated AAV particles as described herein for the manufacture of a medicament. Particular medical conditions wherein the objects of the invention may be used for include proliferative diseases (i.e. cancer) and tissue specific diseases (e.g. liver diseases). It is evident that products described herein interrelated to the conjugated AAV particles (such as the AAV capsid proteins described herein, the nucleic acids described herein, and the nucleic acid vectors described herein) may also be used for medical purposes.

The terms “subject”, “patient”, and “subject in need” may be used interchangeably and refer to animals, preferably warm-blooded animals, more preferably vertebrates, and even more preferably mammals specifically including humans and non-human mammals. The term “mammals”, or “mammalian subjects” refers to any animal classified as such and hence include, but are not limited to humans, domestic animals, commercial animals, farm animals, zoo animals, sport animals, pets and experimental animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. Preferred patients are human subjects. Particularly preferred are human subjects, including both genders and all age categories thereof.

A related aspect of the invention is directed to a method of producing a conjugated AAV particle, which comprise expressing nucleic acids encoding the capsid proteins as described herein in a cell, allowing the cell to form the AAV particles, retrieving the AAV particles and conjugating these to a molecule of interest. In particular embodiments, the methods comprise the steps of
contacting cells with one or more nucleic acids encoding a AAV capsid protein wherein one or more of the VP1-VP2 transition region, VR-I region, VR-IV region or VR-VIII region comprise a sortase recognition sequence;
allowing the cell to assemble a plurality of AAV capsid proteins into an AAV particle and collecting AAV particles from said cells therefrom; and
contacting the AAV particles with a sortase and a heterologous conjugate molecule.

A skilled person appreciates that between the step of providing the cells with the one or more nucleic acids encoding a plurality of AAV capsid proteins and the step of collecting the AAV particles a step of culturing said cells occurs in order to allow transcription and translation of the AAV capsid proteins and assembly into an AAV particle. Optionally, the one or more nucleic acid sequences recited in the method correspond to three nucleic acid sequences: a first nucleic acid encoding the rep and cap genes, a second nucleic acid comprising a transgene and ITR regions, and a third nucleic acid encoding any helper proteins for assembly of the AAV particle. In certain embodiments, the step of collecting AAV particles is preceded by a lysis step of the cells contained in the cell culture. Production methods of AAV particles have been described at numerous occasions in the art and are therefore known to a skilled person.

In preferred embodiments, the sortase recognition sequence is a sortase A recognition sequence, more preferably an Xn-LPXTG-Xm [SEQ ID NO: 10] sequence.

In preferred embodiments, the AAV capsid proteins comprise a sortase recognition sequence in one or more of the VR-I region, VR-IV region or VR-VIII region.

Optionally, before contacting the AAV particles with a sortase and a heterologous conjugate molecule, the method further comprises an additional step of enriching, purifying, and/or isolating assembled AAV particles. Alternatively and/or additionally to the foregoing embodiment, before contacting the AAV particles with a sortase and a heterologous conjugate molecule the method further comprises a step of depleting or removing AAV capsid proteins not part of an assembled AAV particle.

In certain embodiments, in the conjugation step of the methods envisaged herein, the AAV capsid proteins, sortase, and heterologous conjugate molecule are provided in a 1:1:1 ratio. In alternative embodiments, an excess of sortase and heterologous conjugate molecule are provided such that a 1:>1:>1 ratio of molecules is maintained. In yet further embodiments, an excess of heterologous conjugate molecule is provided such that a 1:1:>1 ratio, preferably a 1:1:>5 ratio, more preferably a 1:1>10 ratio is maintained. In preferred embodiments the AAV capsid proteins, sortase, and heterologous conjugate molecule are provided in a ratio of about 1:0.1:1.

Optionally, after contacting the AAV particles with a sortase and a heterologous conjugate molecule the method comprises a further step of enriching, purifying, and/or isolating assembled conjugated AAV particles that contain at least one conjugated AAV capsid protein. In further embodiments, the method comprises a further step of enriching, purifying, and/or isolating assembled conjugated AAV particles that contain at least 10%, preferably at least 20%, preferably at least 30%, preferably at least 40%, preferably at least 50%, preferably at least 60%, preferably at least 70%, preferably at least 80%, preferably at least 90%, or even 100% conjugated AAV capsid protein.

Optionally, the AAV capsid proteins comprise a plurality of distinct sortase recognition sequences and the AAV particles are contacted with different sortases and conjugation molecules in a sequential manner. The plurality of distinct sortase recognition sequences may be provided in a single AAV capsid protein, or may alternatively be provided by using a collection of AAV capsid proteins that each comprise a different sortase recognition sequence, optionally at a distinct location in the AAV capsid protein. Preferably AAV capsid proteins are provided that comprise a sortase recognition sequence in one or more of the VP1-VP2 transition region, VR-I region, VR-IV region or VR-VIII region. In certain embodiments, a combination of naturally occurring AAV capsid proteins and AAV capsid proteins comprising a sortase recognition sequence in one or more of the VP1-VP2 transition region, VR-I region, VR-IV region or VR-VIII region are provided.

It is evident that any embodiment described herein with respect to the AAV capsid protein as such is equally applicable to the (isolated) AAV capsid protein as such but also to AAV capsid protein encoded by a nucleic acid or nucleic acid vector, and (assembled) AAV particles and vice versa.

In addition to the embodiments described herein above, the invention further relates to an adeno-associated virus (AAV) VP1, VP2 or VP3 capsid protein characterized in that in one or more of the VP1-VP2 transition region, VR-I region, VR-IV region and VR-VIII region a sortase recognition sequence Xn-LPXTG-Xm [SEQ ID NO:10] is inserted, wherein n and m range from 0 to 20, and wherein X is any natural amino acid. In particular embodiments, the adeno-associated virus (AAV) VP1, VP2 or VP3 capsid protein is characterized in that in one or more of the VP1-VP2 transition region, VR-I region, VR-IV region and VR-VIII region a sortase recognition sequence Xn-LPXTG-Xm [SEQ ID NO:10] is inserted, wherein n and m range from 0 to 20, and wherein X is any natural amino acid wherein the VP1-VP2 transition region is defined by SEQ ID NO:1 [PVKTAP], the VR-I region is defined by SEQ ID NO:2 [SSQSGASN], the VR-IV region is defined by SEQ ID NO:3 [SRTNTPSGTTTQSRLQFSQAGASDIRDQS] and the VR-VIII region is defined by SEQ ID NO:4 [QYGSVSTNLQRGNRQAATADVNTQGV]. In particular embodiments, the insertion in the VR-IV region is in the fragment with SEQ ID NO:5 [TPSGTTTQS] and/or in the fragment with SEQ ID NO: 6 [LQRGNRQAA]. In particular embodiments, one or more amino acids of the region wherein the sortase recognition site is inserted, are deleted. In particular embodiments of any of the embodiments above, the AAV is AAV2, or AAV9. In further particular embodiments of any of the embodiments above, n is between 15 and 20, between 10 and 15, between 5 and 10, between 0 or 5, or is 10, 9, 8, 7, 6, 5, 4, 3, 2, or 1. Particularly it is envisaged that m is between 15 and 20, between 10 and 15, between 5 and 10, between 0 or 5, or is 10, 9, 8, 7, 6, 5, 4, 3, 2, or 1 amino acid. In particular embodiments of any of the embodiments above, the linker sequence Xn or Xm comprises for least 80% of Gly, Ser, Thr and Ala. Preferably, the linker sequence Xm or Xn consist of amino acids selected from Gly, Ser, Thr and Ala. In particular embodiments of any of the embodiments above, X in LPXTG is Asp, Glu, Ans or Gln, typically Gln. In particular embodiments of any of the embodiments above, the sortase recognition sequence LPXTG [SEQ ID NO:10] is inserted in the VR-1 region and one or more of Lysines at positions K258, K321, K490, K507, K527, K532, K544, K549, K556, K620, K640, K649, K665, K692, K706 are mutated. In further particular embodiments, the sortase recognition sequence LPXTG [SEQ ID NO:10] is inserted in the VR-1 region and one or more of Lysines at positions K258, K507, K527, K549 and K706 are mutated. In further particular embodiments the sortase recognition sequence LPXTG [SEQ ID NO: 10] is inserted in the VR-IV region and wherein one or more of Lysines at position K258, K309, K313, K321, K490, K507, K527, K532, K544, K549, K556, K620, K640, K649, K665, K688, K692 and K706 are mutated. In further particular embodiments, the sortase recognition sequence LPXTG [SEQ ID NO:10] is inserted in the VR-IV and wherein one or more of Lysines at position K258, K490, K507, K532, K544, K549, K556 and K665 are mutated. In further particular embodiments, the sortase recognition sequence LPXTG [SEQ ID NO:10] is inserted in the VR-IV and wherein Lysine at position K549 are mutated. In further particular embodiments, the sortase recognition sequence LPXTG [SEQ ID NO:10] is inserted in the VR-VIII and wherein one or more of Lysines at position K309, K490, K507, K527, K532, K544, K549, K556, K620, K640, K688 and K706 are mutated. In further particular embodiments, the sortase recognition sequence LPXTG [SEQ ID NO:10] is inserted in the VR-VIII and wherein one or more Lysines at position K490, K507, K527 and K532 are mutated. In further particular embodiments, the sortase recognition sequence LPXTG [SEQ ID NO:10] is inserted in the VR-VIII and wherein one or more of Lysines at positions K507 are mutated into Gly or Ala. In preferred embodiments of the embodiments described herein, said one or more lysines are mutated into Gly, Ser or Ala. The invention also provides a nucleic acid encoding the capsid protein of any one of the embodiments as described herein above. The invention further provides an expression vector comprising these nucleic acids. The invention further provides an AAV particle comprising a AAV capsid protein as described herein above. In particular embodiments, the ratio of unmodified AAV protein over modified protein is between 1/20, 1/10 or 1/5 and 5/1, 10/1 or 20/1. In particular embodiments, the AAV particle is fused via the sortase recognition site to a conjugate such as a small molecule, carbohydrate, polypeptide. In further particular embodiments, the AAV particle is fused via the sortase recognition site to a targeting moiety such as is a ligand of cell receptor, or a protein binding to a cell surface protein. In further particular embodiments, the protein binding to a cell surface protein is an antibody or a nanobody, such as an antibody or nanobody that binds HER2. The invention further provides for the use of an AAV particle as described herein, such as the AAV particle as described herein for use as a medicament.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects, statements, and embodiments of the invention are further supported by the following non-limiting examples.

### EXAMPLES

### Example 1: General Design Information, Considerations, and Explorative Results

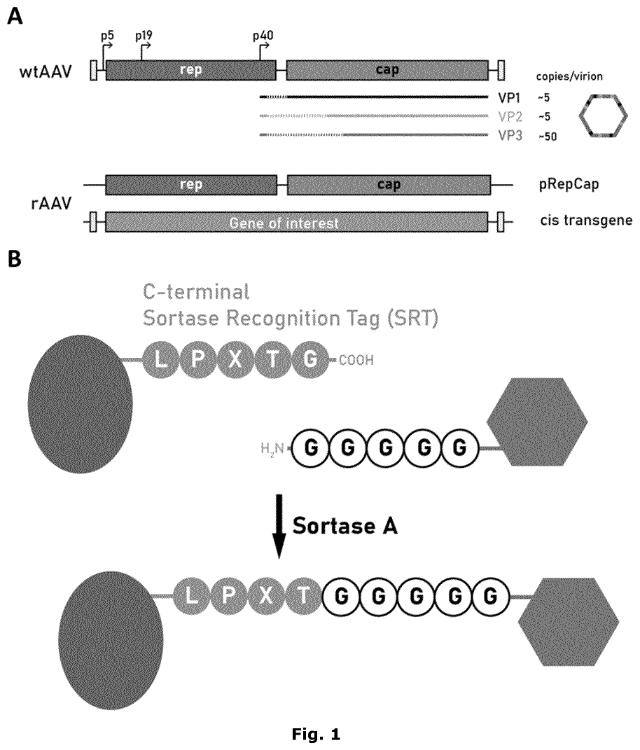

Adeno-associated virus (AAV) is a small, non-pathogenic, non-enveloped ssDNA virus. Its genome (4.7 kb) consists of two major open reading frames. The rep gene encodes 4 replication proteins; the cap gene codes for the three structural proteins of the AAV capsid (VP1, VP2 and VP3) which are formed through alternative splicing and alternative start codons. In a 1:1:10 ratio, they form the 60-subunit capsid coat. Flanking the rep and cap genes are the inverted terminal repeats (ITRs), which are the only genetic elements required for viral DNA replication and packaging. As a result, the viral rep and cap genes can be provided in trans during the production process and transgenes of interest of ˜4.5 kb can be inserted between the ITRs, resulting in recombinant AAV (rAAV) viral vectors (FIG. 1A).

Sortase reactions in general have been described at numerous instances throughout the art and are therefore known to a person of ordinary skill in the art. A general overview of a sortase A reaction is depicted in FIG. 1B. This results in an efficient and controlled antibody-drug coupling with homogenous and predictable drug to antibody ratio. This technology has so far not been used for enzymatic coupling of targeting moieties to AAV vectors.

The potential of targeting AAV vectors is dependent on the targeting potential of the ligand. Nanobodies are a preferred moiety suited for this. They combine a small and compact structure with high specificity, high stability and a low immunogenic profile, and the Sortase A technology has been extensively used for nanobody conjugation to drugs or other nanobodies. Moreover, nanobodies can be designed against virtually any cellular receptor. The present invention is illustrated with the anti-HER2 (human epidermal growth factor receptor 2) nanobody 2Rs15d, used in breast cancer radionuclide therapy.

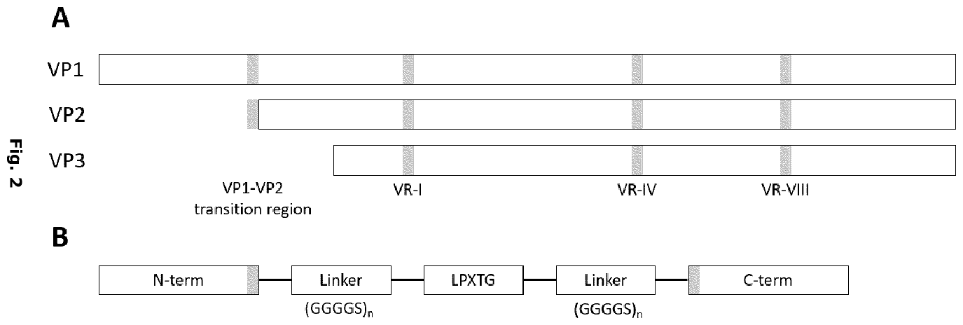

The Sortase A LPXTG recognition motif [SEQ ID NO: 10] can function as part of an exposed loop of the target protein. Previous literature has shown that peptide insertion up to 34 amino acids into variable regions (VR)-IV and VR-VIII of the AAV capsid is well tolerated. Placing the LPXTG recognition motif [SEQ ID NO: 10] in VR-IV and VR-VIII is shown in (FIGS. 2A and 2B). The LPXTG [SEQ ID NO: 10] binding cleft on the Sortase A enzyme is a rather deep binding pocket, so the length and sequence of the LPXTG [SEQ ID NO: 10] flanking linker is considered.

AAV2-HBO, in which the ability to bind the heparan sulphate proteoglycan receptor has been destroyed by the mutations (R585A and R588A) in order to mute the natural tropism of AAV2, is used in the examples of the present invention. However, it is evident that the mutations of this particular AAV2 mutant are not limiting for the context of the invention, and the findings described herein are readily applicable to other AAV serotypes in general and other AAV2 variations.

Sortase A-mediated coupling reaction is performed with an Ab recognition (e.g. HA or FLAG) and Affinity tag (e.g. His) tagged nanobody-protein. The recombinant 2Rs15d nanobody is used in the present examples.

Different constructs are tested in an experiment to assess ligand binding using Sortase A. The LPQTG motif [SEQ ID NO: 43] with optional linkers was inserted as shown in Table 1.

TABLE 1
Sequence information regarding tested insertion sites.

| Capsid/Region/ | Flanking sequence (5 AA) (lower case) and |
|---|---|
| amino acid | Inserted sequence (capital) |
| VP1/VR-IV/453-454 | ntpsg-LPQTG-tttqs [SEQ ID NO: 36] |
| VP1/VR-VIII/587-588 | Iqrgn-GGSGS LPQTG GGSGS-rqaat [SEQ ID NO: 37] |
| VP1/VR-VIII/587-588 | lqrgn-GGSGS GGSGS LPQTG GGSGS GGSGS-rqaat |
| | [SEQ ID NO: 38] |

The nanobody is typically N-terminally tagged with a Gly₅ tag and C-terminally fused with a 3×FLAG-His₆ for nanobody production. Unconjugated and undesired products are removed using diafiltration.

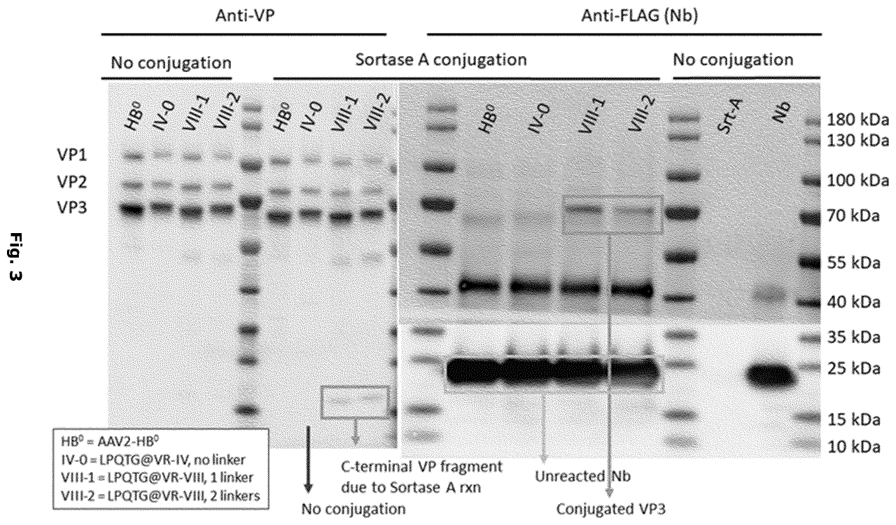

AAV2 tolerates LPQTG [SEQ ID NO: 43] insertion into loop IV and VIII with different linker lengths. The LPQTG motif [SEQ ID NO: 43] was inserted into AAV2-HBO VR-IV (between AA 453 and 454) VR-VIII (between AA 587 and 588), without linker or flanked by 1 or 2 GGSGS [SEQ ID NO: 47] repeats at either side of the motif. Next, a Gly5-nanobody was conjugated using Sortase A. Western blots were ran, and imaged with an anti-VP antibody (detecting VP1, 2 and 3) and an anti-FLAG antibody (detecting the nanobody). As the Sortase A reaction cleaves the VP primary structure during conjugation, a 17-18 kDa band is expected and seen in VIII-1 and VIII-2 (FIG. 3).

### Example 2: AAV2_VR-I 0-2L

### Materials and Methods

LPQTG-containing AAV vectors (SEQ ID NO: 13, SEQ ID NO: 14, SEQ ID NO: 15) [AAV2-HBO with an LPQTG tag located at position 265 with 0, 1 or 2 GGSGS linkers flanking both sides] with Sortase A and anti-GFP nanobody (SEQ ID NO: 11) in 1× Sortase reaction buffer (300 mM Tris, 150 mM NaCl, 5 mM CaCl2, pH 7.5). The molar concentration of Sortase A was 100-fold higher than the molar concentration of AAV viral proteins in the mixture, while the nanobody was present in a 100-fold higher molar concentration relative to the AAV viral proteins. The mixes were incubated for 16 hours at 25° C. The reacted samples, together with an equal amount of vector in Sortase reaction buffer without Sortase A or nanobody, were then ran on an SDS-PAGE gel and blotted to a PVDF membrane. After blocking, the membrane was incubated with 1:1000 rabbit anti-VP1/2/3 (Progen #61084) at 4° C. for 16 h. After washing, the gel was incubated for 1 h at RT with 1:10000 HRP-conjugated goat anti-rabbit antibody (Dako P0448) and developed using chemiluminescence.

In parallel, an equal amount of unreacted nanobody was loaded on a separate SDS-PAGE gel, which was blotted to a PVDF membrane and stained with 1:1000 mouse anti-FLAG antibody (Novus Bio NBP1-97410) for 16 h at 4° C. After washing, the gel was incubated for 1 h at RT with 1:10000 HRP-conjugated goat anti-mouse antibody (Dako P0447) and developed using chemiluminescence.

### Results and Discussion

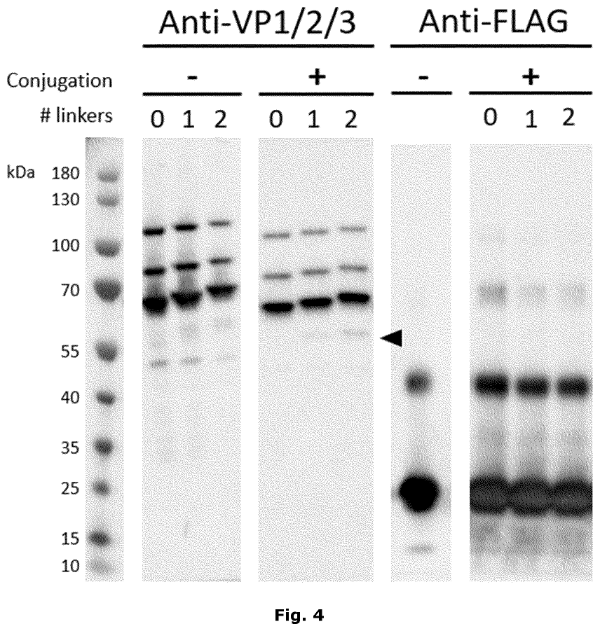

In the anti-VP1/2/3 stained blot with nanobody-conjugated samples (FIG. 4), a faint band just above the 55 kDa reference band is visible (arrow), with increasing intensity as the linker length increases from 0 to 1 to 2 GGSGS [SEQ ID NO: 47] elements. This apparent molecular mass is consistent with a Sortase A-mediated backbone cleavage, which yields a 53-kDa C-terminal VP fragment.

Similarly, a faint band just above the 35 kDa marker is visible, which might be pointing towards a nanobody-conjugated VP1 or VP2 fragment. However, the 100-fold excess of nanobody and the unknown 40 kDa species in the nanobody prep make identification of the more faint bands difficult.

In conclusion, the LPQTG tag [SEQ ID NO: 43] can be inserted in VR-I, and conjugation is less efficient. Conjugation efficiency increases with increasing linker length, though is in general less efficient at this insertion site.

### Example 3: AAV2_VR-IV 0-5L

### Materials and Methods

LPQTG-containing AAV vectors (SEQ ID NO: 16 to SEQ ID NO: 20) [AAV2-HBO with an LPQTG tag located at position 453 with 0, 1, 3, 4 or 5 GGSGS linkers flanking both sides] with Sortase A and anti-GFP nanobody (SEQ ID NO: 11) in 1× Sortase reaction buffer (300 mM Tris, 150 mM NaCl, 5 mM CaCl2, pH 7.5). The molar concentration of Sortase A was 10-fold higher than the molar concentration of AAV viral proteins in the mixture, while the nanobody was present in a 100-fold higher molar concentration relative to the AAV viral proteins. Identical reaction mixes, though without Sortase A and without nanobody, were also made. The reaction mixes were incubated for 16 hours at 25° C. The samples were then ran on an SDS-PAGE gel and blotted to a PVDF membrane. After blocking, the membrane was incubated with 1:1000 rabbit anti-VP1/2/3 (Progen #61084) at 4° C. for 16 h. After washing, the gel was incubated for 1 h at RT with 1:10000 HRP-conjugated goat anti-rabbit antibody (Dako P0448) and developed using chemiluminescence.

In parallel, the reaction mixtures were loaded on a separate SDS-PAGE gel, which was blotted to a PVDF membrane and stained with 1:1000 mouse anti-FLAG antibody (Novus Bio NBP1-97410) for 16 h at 4° C. After washing, the gel was incubated for 1 h at RT with 1:10000 HRP-conjugated goat anti-mouse antibody (Dako P0447) and developed using chemiluminescence.

### Results and Discussion

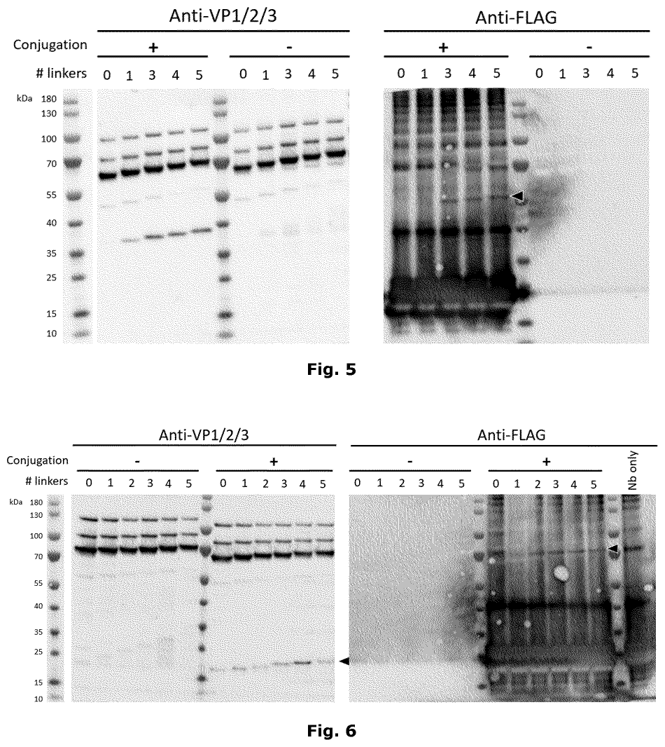

In the anti-VP1/2/3 stained blot with nanobody-conjugated samples, clear bands at 37-39 kDa are clearly visible (FIG. 5). The intensity increases from 0 to 1 to 3 linker repeats, and seems to stabilize as the linker length increases to 4 and 5 linker repeats. This apparent molecular mass is, given that most fragments migrate slightly higher than their expected molecular weight, consistent with a Sortase A-mediated backbone cleavage, which yields 32-35 kDa C-terminal VP fragments.

Similarly, a band is visible around the 55 kDa marker, which is indicative of the nanobody-conjugated N-terminal VP fragment. This band is not visible in the absence of a linker fragment, barely there when there is one linker repeat visible, but clearly there at 3, 4, or 5 linker repeats, with no apparent difference in intensity between 3, 4, or 5 repeats.

In conclusion, the LPQTG tag [SEQ ID NO: 43] can be inserted in VR-IV in AAV2, with good conjugation efficiency. Conjugation efficiency increases with increasing linker length, and seems to plateau at 3 GGSGS linker repeats on each side of the LPQTG tag.

### Example 4: AAV2_VR-VIII 0-5L

### Materials and Methods

LPQTG-containing AAV vectors (SEQ ID NO: 21 to SEQ ID NO: 26) [AAV2-HBO with an LPQTG tag located at position 587 with 0, 1, 2, 3, 4 or 5 GGSGS linkers flanking both sides] with Sortase A and anti-GFP nanobody (SEQ ID NO: 11) in 1× Sortase reaction buffer (300 mM Tris, 150 mM NaCl, 5 mM CaCl2, pH 7.5). The molar concentration of Sortase A was 10-fold higher than the molar concentration of AAV viral proteins in the mixture, while the nanobody was present in a 100-fold higher molar concentration relative to the AAV viral proteins. Identical reaction mixes, though without Sortase A and without nanobody, were also made. The reaction mixes were incubated for 16 hours at 25° C. The samples were then ran on an SDS-PAGE gel and blotted to a PVDF membrane. After blocking, the membrane was incubated with 1:1000 rabbit anti-VP1/2/3 (Progen #61084) at 4° C. for 16 h. After washing, the gel was incubated for 1 h at RT with 1:10000 HRP-conjugated goat anti-rabbit antibody (Dako P0448) and developed using chemiluminescence.

In parallel, the reaction mixtures were loaded on a separate SDS-PAGE gel, together with a sample containing an identical amount of nanobody only. The which was blotted to a PVDF membrane and stained with 1:1000 mouse anti-FLAG antibody (Novus Bio NBP1-97410) for 16 h at 4° C. After washing, the gel was incubated for 1 h at RT with 1:10000 HRP-conjugated goat anti-mouse antibody (Dako P0447) and developed using chemiluminescence.

### Results and Discussion

In the anti-VP1/2/3 stained blot with nanobody-conjugated samples, clear bands at 17-20 kDa are clearly visible (FIG. 6). The intensity increases from 0 to 1 to 3 to 4 linker repeats (with 2 linker repeats being lower in intensity), and seems to be lower again at 5 linker repeats. This apparent molecular mass is consistent with a Sortase A-mediated backbone cleavage, which yields 17-20 kDa C-terminal VP fragments.

Similarly, a band is visible around the 70 kDa marker, which is indicative of the nanobody-conjugated N-terminal VP fragment, though migrating slightly higher, as is the case for the unreacted VP1/2/3 bands. This band is not visible in the absence of a linker fragment, increases from 1 to 2 to 3 to 4 linker repeats, and is again slightly lower for 5 linker repeats.

In conclusion, the LPQTG tag [SEQ ID NO: 43] can be inserted in VR-VIII in AAV2, with moderate conjugation efficiency. Conjugation efficiency increases with increasing linker length, and seems to peak at 4 GGSGS [SEQ ID NO: 47] linker repeats on each side of the LPQTG tag [SEQ ID NO: 43].

### Example 5: AAV9_VR-IV 0-3L

### Materials and Methods

LPQTG-containing AAV vectors (SEQ ID NO: 27 to SEQ ID NO: 30) [AAV9-A with an LPQTG tag located at position 455 with 0, 1, 2 or 3 GGSGS linkers flanking both sides] with Sortase A and anti-GFP nanobody (SEQ ID NO: 11) in 1× Sortase reaction buffer (300 mM Tris, 150 mM NaCl, 5 mM CaCl2, pH 7.5). The molar concentration of Sortase A was 10-fold higher than the molar concentration of AAV viral proteins in the mixture, while the nanobody was present in a 100-fold higher molar concentration relative to the AAV viral proteins. The mixes were incubated for 16 hours at 25° C. The reacted samples, together with an equal amount of vector in Sortase reaction buffer without Sortase A or nanobody, were then ran on an SDS-PAGE gel and blotted to a PVDF membrane. After blocking, the membrane was incubated with 1:1000 mouse anti-VP1/2/3 (Progen #65158) at 4° C. for 16 h. After washing, the gel was incubated for 1 h at RT with 1:10000 HRP-conjugated goat anti-mouse antibody (Dako P0447) and developed using chemiluminescence.

In parallel, an equal amount of unreacted AAV and an equal amount of unreacted nanobody was loaded on a separate SDS-PAGE gel, which was blotted to a PVDF membrane and stained with 1:1000 mouse anti-FLAG antibody (Novus Bio NBP1-97410) for 16 h at 4° C. After washing, the gel was incubated for 1 h at RT with 1:10000 HRP-conjugated goat anti-mouse antibody (Dako P0447) and developed using chemiluminescence.

### Results and Discussion

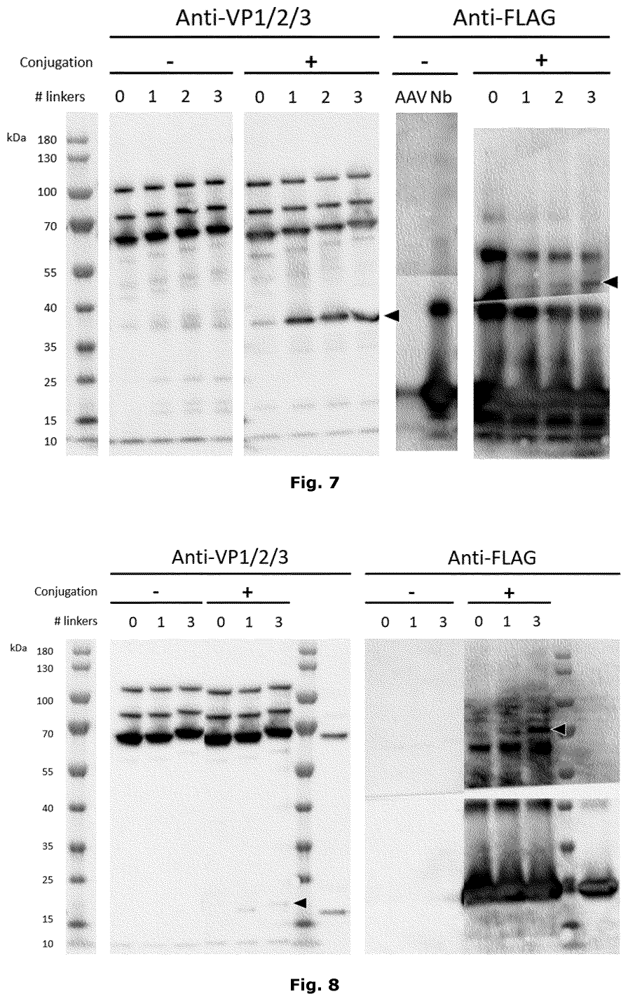

In the anti-VP1/2/3 stained blot with nanobody-conjugated samples, clear bands at 37 kDa are clearly visible (FIG. 7). The intensity increases from 0 to 1 linker repeats and 1, 2 and 3 linker repeats have similar intensity. This apparent molecular mass is, given that most fragments migrate slightly higher than their expected molecular weight, consistent with a Sortase A-mediated backbone cleavage, which yields 33 kDa C-terminal VP fragments.

In the anti-FLAG gel, a band is visible at 45 kDa, which is indicative of the nanobody-conjugated N-terminal VP fragment. This band is not visible in the absence of a linker fragment, but increases in intensity from 1 to 2 to 3 linker repeats.

In conclusion, the LPQTG tag [SEQ ID NO: 43] can be inserted in VR-IV in AAV9, with good conjugation efficiency. Conjugation efficiency increases with increasing linker length, and reaches its highest value at 3 GGSGS [SEQ ID NO: 47] linker repeats on each side of the LPQTG tag [SEQ ID NO: 43], without longer linker lengths being tested with this construct.

### Example 6: AAV9_VR-VIII 0-1-3L

### Materials and Methods

LPQTG-containing AAV vectors (SEQ ID NO: 31, SEQ ID NO: 32, SEQ ID NO: 33) [AAV9-A with an LPQTG tag located at position 589 with 0, 1, or 3 GGSGS linkers flanking both sides] with Sortase A and anti-GFP nanobody (SEQ ID NO: 11) in 1× Sortase reaction buffer (300 mM Tris, 150 mM NaCl, 5 mM CaCl2, pH 7.5). The molar concentration of Sortase A was 10-fold higher than the molar concentration of AAV viral proteins in the mixture, while the nanobody was present in a 100-fold higher molar concentration relative to the AAV viral proteins. The mixes were incubated for 16 hours at 25° C. The reacted samples, together with a sample without Sortase A, were then ran on an SDS-PAGE gel and blotted to a PVDF membrane. After blocking, the membrane was incubated with 1:1000 mouse anti-VP1/2/3 (Progen #65158) at 4° C. for 16 h. After washing, the gel was incubated for 1 h at RT with 1:10000 HRP-conjugated goat anti-mouse antibody (Dako P0447) and developed using chemiluminescence.

In parallel, we loaded and ran the reaction mixture, together with a sample without Sortase A, on a separate SDS-PAGE gel, which was blotted to a PVDF membrane and stained with 1:1000 mouse anti-FLAG antibody (Novus Bio NBP1-97410) for 16 h at 4° C. After washing, the gel was incubated for 1 h at RT with 1:10000 HRP-conjugated goat anti-mouse antibody (Dako P0447) and developed using chemiluminescence.

### Results and Discussion

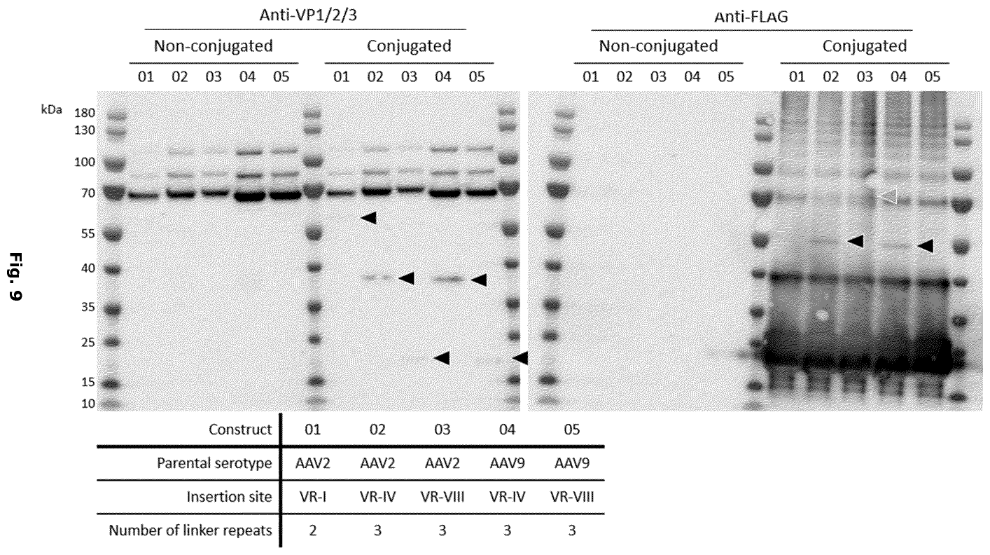

In the anti-VP1/2/3 stained blot with nanobody-conjugated samples, faint bands at 17 kDa are visible (FIG. 8). The band is indiscernible with 1 linker repeat, but intensity increases from 1 to 3 linker repeats. This apparent molecular mass is consistent with a Sortase A-mediated backbone cleavage, which yields 17 kDa C-terminal VP fragments.

In the anti-FLAG gel, a band is visible at 70 kDa, which is indicative of the nanobody-conjugated N-terminal VP fragment. This band is extremely faint in the absence of a linker fragment, but increases in intensity from 1 to 3 linker repeats.

In conclusion, the LPQTG [SEQ ID NO: 43] tag can be inserted in VR-VIII in AAV9, but conjugation is inefficient. Conjugation efficiency increases with increasing linker length, and reaches its highest value at 3 GGSGS [SEQ ID NO: 47] linker repeats on each side of the LPQTG tag [SEQ ID NO: 43], without longer linker lengths being tested with this construct.

### Example 7: AV2_VR-I 2L, AAV2 VR-IV 3L, AAV2 VR-VIII 3L, AAV9_VR-IV 3L, and AAV2_VR-VIII 3L

### Materials and Methods

LPQTG-containing AAV vectors (SEQ ID NO: 15, SEQ ID NO: 18, SEQ ID NO: 24, SEQ ID NO: 30, SEQ ID NO: 33) [AAV2-HBO with an LPQTG tag located at position 265 with 2 GGSGS linkers flanking both sides, AAV2-HBO with an LPQTG tag located at position 453 with 3 GGSGS linkers flanking both sides, AAV2-HBO with an LPQTG tag located at position 587 with 3 GGSGS linkers flanking both sides, AAV9-A with an LPQTG tag located at position 455 with 3 GGSGS linkers flanking both sides and AAV9-A with an LPQTG tag located at position 589 with 3 GGSGS linkers flanking both sides] were incubated with Sortase A and anti-HER2 nanobody (SEQ ID NO: 12) in 1× Sortase reaction buffer (300 mM Tris, 150 mM NaCl, 5 mM CaCl2, pH 7.5). The molar concentration of Sortase A was 10-fold higher than the molar concentration of AAV viral proteins in the mixture, while the nanobody was present in a 100-fold higher molar concentration relative to the AAV viral proteins. The mixes were incubated for 16 hours at 25° C. The reacted samples, together with an equal amount of vector in Sortase reaction buffer without Sortase A or nanobody, were then ran on an SDS-PAGE gel and blotted to a PVDF membrane. After blocking, the membrane was incubated with 1:1000 rabbit anti-VP1/2/3 (Progen #61084) at 4° C. for 16 h. After washing, the gel was incubated for 1 h at RT with 1:10000 HRP-conjugated goat anti-rabbit antibody (Dako P0448) and developed using chemiluminescence. In parallel, the reaction mixtures were loaded on a separate SDS-PAGE gel, which was blotted to a PVDF membrane and stained with 1:1000 mouse anti-FLAG antibody (Novus Bio NBP1-97410) for 16 h at 4° C. After washing, the gel was incubated for 1 h at RT with 1:10000 HRP-conjugated goat anti-mouse antibody (Dako P0447) and developed using chemiluminescence.

### Results and Discussion

In the anti-VP1/2/3 stained blot, several band relating to the Sortase A-generated C-terminal VP fragment are visible (FIG. 9). For AAV2_VR-I, the band at ˜58 kDa is fairly faint, pointing to inefficient coupling. For AAV2_VR-IV and AAV9_VR-IV, the band at ˜38 kDa is clearly visible, indicating efficient coupling. For AAV2_VR-VIII and AAV9_VR-VIII, the band at ˜18 kDa is visible at higher intensity that AAV2_VR-I but less intense then AAV2_VR-IV and AAV9_VR-IV, indicating intermediate coupling.

The same findings are reflected in the FLAG-stained blot. The bands corresponding to the conjugated AAV2_VR-I and AAV9-VR-VIII are fully obscured by the background from the nanobody. The band corresponding to the conjugated AAV2_VR-VIII is very faintly visible. The bands corresponding to AAV2_VR-IV and AAV9_VR-IV are clearly visible.

In conclusion, insertions into VR-IV display highest coupling efficiency. Insertions into VR-VIII do seem to work, though at a lower efficiency and insertions into VR-I are possible, though with very low relative efficiency.

### Example 8: GGG-Biotin

### Materials and Methods

LPQTG-containing AAV vector (SEQ ID NO: 22 (587_1L_pdAAVe_008)) [AAV2-HBO with an LPQTG tag located at position 587 with 1 GGSGS linker flanking both sides] was incubated with Sortase A and a biotin-tagged GGG peptide (GGG-[K(Biotin)]-amide, Cambridge Research Biochemicals, crb1000649h) in 1× Sortase reaction buffer (300 mM Tris, 150 mM NaCl, 5 mM CaCl2, pH 7.5). The molar concentration of Sortase A was 100-fold higher than the molar concentration of AAV viral proteins in the mixture, while the GGG-biotin peptide was present in a 250-fold higher molar concentration relative to the AAV viral proteins (sample (1)). Control samples included (2) “no AAV”, (3) no Sortase A, (4) no GGG-biotin peptide, (5) GGG-biotin peptide only, (6) GGG-biotin peptide with BSA and (7) BSA only. In the control reactions, all components were at the same concentration as in reaction (1), and the mass amount of BSA in sample (6) and (7) was identical to the mass amount of viral proteins in sample (1). The reaction mixtures were incubated for 16 hours at 25° C. The reacted samples were then ran on an SDS-PAGE gel and blotted to a PVDF membrane. After blocking, the membrane was incubated with 1:10 000 streptavidin-HRP (ThermoScientific, 21130) and developed using chemiluminescence. As a reference for the VPs' molecular weight on gel, a sample with AAV only, blotted and imaged using an anti-VP1/2/3 antibody, was included from a different blot.

### Results and Discussion

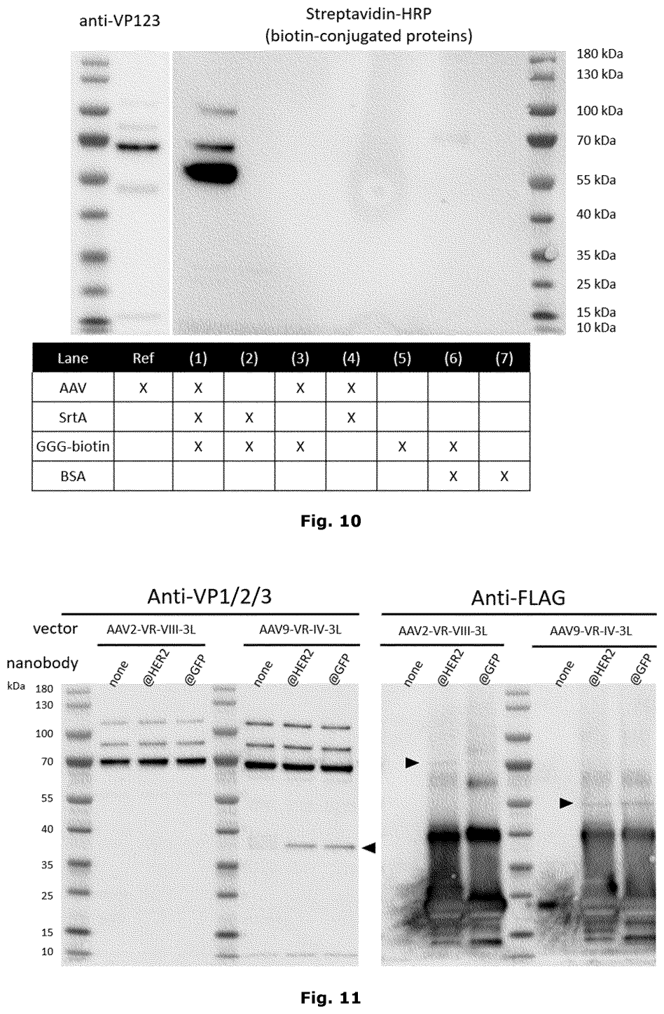

In the Streptavidin-HRP blot, Sortase A-mediated biotin labeling of VP1, 2 and 3 can be observed in sample (1), with all negative controls showing no sign of conjugation (FIG. 10). The molecular masses are lower than the non-conjugated VPs, which is expected given the removal of a 17 kDa C-terminal fragment.

### Example 9: HER2 vs GFP

### Materials and Methods

LPQTG-containing AAV vectors (SEQ ID NO: 24, SEQ ID NO: 30) [AAV2-HBO with an LPQTG tag located at position 453 with 3 GGSGS linkers flanking both sides, AAV9-A with an LPQTG tag located at position 589 with 3 GGSGS linkers flanking both sides] were incubated with Sortase A and anti-HER2 nanobody (SEQ ID NO: 12), anti-GFP nanobody (SEQ ID NO: 11) or no nanobody in 1× Sortase reaction buffer (300 mM Tris, 150 mM NaCl, 5 mM CaCl2, pH 7.5). The molar concentration of Sortase A was 10-fold higher than the molar concentration of AAV viral proteins in the mixture, while the nanobody was present in a 100-fold higher molar concentration relative to the AAV viral proteins. The mixes were incubated for 16 hours at 25° C. The reacted samples were then ran on an SDS-PAGE gel and blotted to a PVDF membrane. After blocking, the membrane was incubated with 1:1000 rabbit anti-VP1/2/3 (Progen #61084) at 4° C. for 16 h. After washing, the gel was incubated for 1 h at RT with 1:10000 HRP-conjugated goat anti-rabbit antibody (Dako P0448) and developed using chemiluminescence.

In parallel, the reaction mixtures were loaded on a separate SDS-PAGE gel, which was blotted to a PVDF membrane and stained with 1:1000 mouse anti-FLAG antibody (Novus Bio NBP1-97410) for 16 h at 4° C. After washing, the gel was incubated for 1 h at RT with 1:10000 HRP-conjugated goat anti-mouse antibody (Dako P0447) and developed using chemiluminescence.

### Results and Discussion

As observed in the previous experiments, Sortase A-mediated nanobody conjugation of AAV2 with an LPQTG [SEQ ID NO: 43] insertion at position 587, flanked with 3 GGSGS [SEQ ID NO: 47] linker repeats on each side, is visible but inefficient (FIG. 11). Sortase A-mediated nanobody conjugation of AAV9 with an LPQTG [SEQ ID NO: 43] insertion at position 455, flanked with 3 GGSGS [SEQ ID NO: 47] linker repeats on each side, is visible with good conjugation efficiency.

Most importantly from this experiment, there does not seem to be a difference in conjugation efficiency between the anti-HER2 and anti-GFP nanobodies.

### Example 10: LPQTG vs LPETG in AAV2_VR-VIII and AAV9_VR-IV

### Materials and Methods

LPQTG-containing AAV vectors (SEQ ID NO: 24, SEQ ID NO: 34, SEQ ID NO: 30, SEQ ID NO: 35) [AAV2-HBO with an LPQTG tag located at position 587 with 3 GGSGS linkers flanking both sides, AAV2-HBO with an LPETG tag located at position 587 with 3 GGSGS linkers flanking both sides, AAV9-A with an LPQTG tag located at position 455 with 3 GGSGS linkers flanking both sides and AAV9-A with an LPETG tag located at position 455 with 3 GGSGS linkers flanking both sides] were incubated with Sortase A and anti-HER2 nanobody (SEQ ID NO: 12) in 1× Sortase reaction buffer (300 mM Tris, 150 mM NaCl, 5 mM CaCl2, pH 7.5). The molar concentration of Sortase A was 5-fold higher than the molar concentration of AAV viral proteins in the mixture, while the nanobody was present in a 50-fold higher molar concentration relative to the AAV viral proteins. The mixes were incubated for 20 hours at 25° C. The reacted samples were then ran on an SDS-PAGE gel and blotted to a PVDF membrane. After blocking, the membrane was incubated with 1:500 rabbit anti-VP1/2/3 (Progen #61084) at 4° C. for 16 h. After washing, the gel was incubated for 1 h at RT with 1:10000 HRP-conjugated goat anti-rabbit antibody (Dako P0448) and developed using chemiluminescence.

In parallel, we loaded and ran the reaction mixtures on a separate SDS-PAGE gel, which was blotted to a PVDF membrane and stained with 1:1000 mouse anti-FLAG antibody (Novus Bio NBP1-97410) for 16 h at 4° C. After washing, the gel was incubated for 1 h at RT with 1:10000 HRP-conjugated goat anti-mouse antibody (Dako P0447) and developed using chemiluminescence.

### Results and Discussion

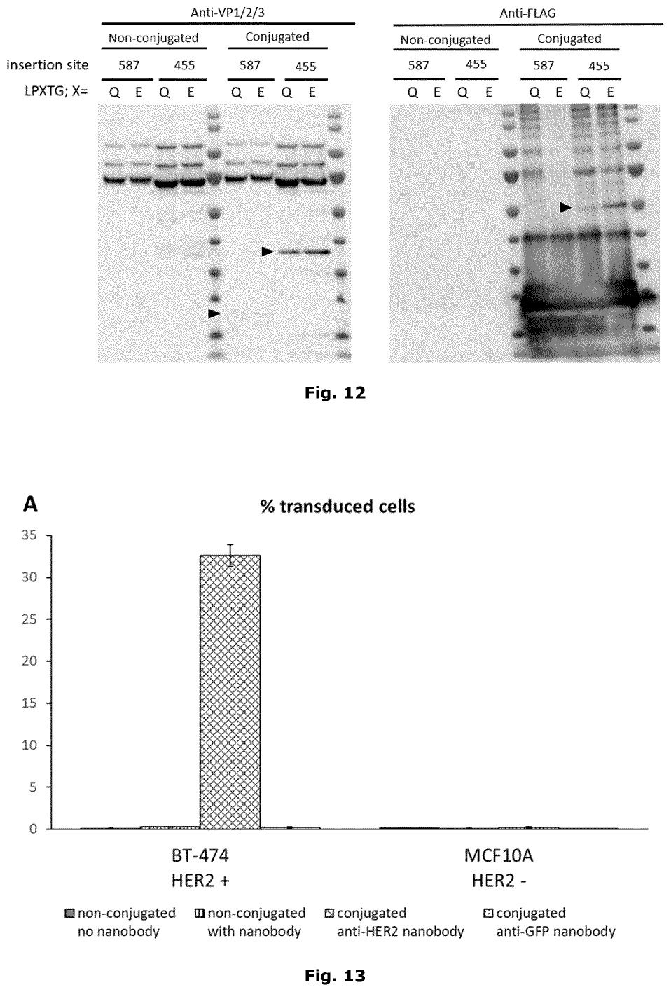

As observed in the previous experiments, Sortase A-mediated nanobody conjugation of AAV2 with an LPQTG insertion at position 587, flanked with 3 GGSGS [SEQ ID NO: 47] linker repeats on each side, is visible but inefficient (FIG. 12). Sortase A-mediated nanobody conjugation of AAV9 with an LPQTG [SEQ ID NO: 43] insertion at position 455, flanked with 3 GGSGS [SEQ ID NO: 47] linker repeats on each side, is visible with good conjugation efficiency.

From both the anti-VP1/2/3 and the anti-FLAG blot, it seems like LPETG [SEQ ID NO: 44] has a slightly higher conjugation efficiency compared to LPQTG [SEQ ID NO: 43], at least for the anti-HER2 nanobody conjugation to AAV9 with the LPXTG [SEQ ID NO: 10] inserted at position 455, flanked with 3GGSGS [SEQ ID NO: 47] linker repeats on each side.

### Example 11: In Vitro Targeting

### Materials and Methods

LPQTG-containing AAV vector (SEQ ID NO: 24) [AAV2-HBO with an LPQTG tag located at position 587 with 3 GGSGS linkers flanking both sides] was incubated with Sortase A and anti-HER2 nanobody (SEQ ID NO: 12) (“Conjugated anti-HER2 Nb”), Sortase A and anti-GFP nanobody (SEQ ID NO: 11) (“Conjugated anti-GFP Nb”), anti-HER2 nanobody (SEQ ID NO: 12) without Sortase A (“non-conjugated with Nb”), and neither nanobody nor Sortase A (“non-conjugated without Nb”) in 1× Sortase reaction buffer (300 mM Tris, 150 mM NaCl, 5 mM CaCl2, pH 7.5). The molar concentration of Sortase A was 10-fold higher than the molar concentration of AAV viral proteins in the mixture, while the nanobody was present in a 100-fold higher molar concentration relative to the AAV viral proteins. The reactions were incubated for 20 hours at 25° C. before being dialyzed using a Micro Float-A-Lyzer 100 kDa MWCO (Repligen, F235049) against PBS to remove Sortase A enzyme and unreacted nanobody. Finally, the viral genome titer was determined using ddPCR.

MCF-10A (HER2-negative) and BT-474 (HER2-positive) cells were cultured in MEBM (Lonza) supplemented with 10% FBS and RPMI1640 (Gibco) medium, supplemented Glutamax and 10% FBS, respectively. Cells were seeded at 50.000 cells in 90 ul of medium, and mixed with vector at an MOI of 1E5 vg/cell in 30 ul. This mixture was seeded in a 96-well plate, and after 3 h, 80 ul of medium was added. After 72 h, cells were washed with PBS, trypsinized, spun down, and re-suspended in 400 ul PBS with 1:10 000 PerCP-Cy5.5-conjugated anti-HER2 antibody (BioLegend 324416). After incubation for 30 min at RT, the cell suspension was analyzed using an Attune flow cytometer.

### Results and Discussion

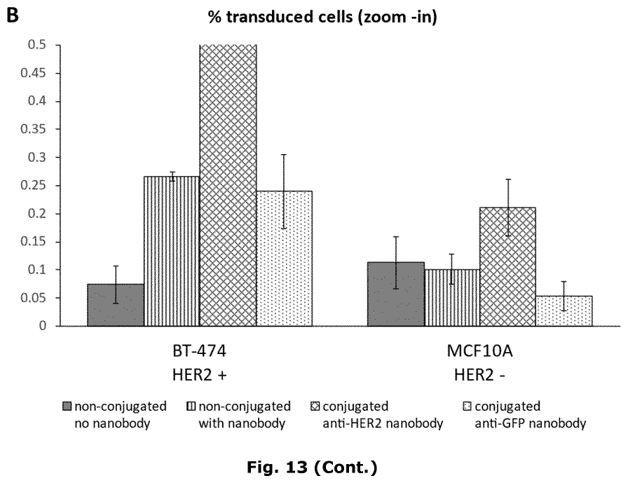

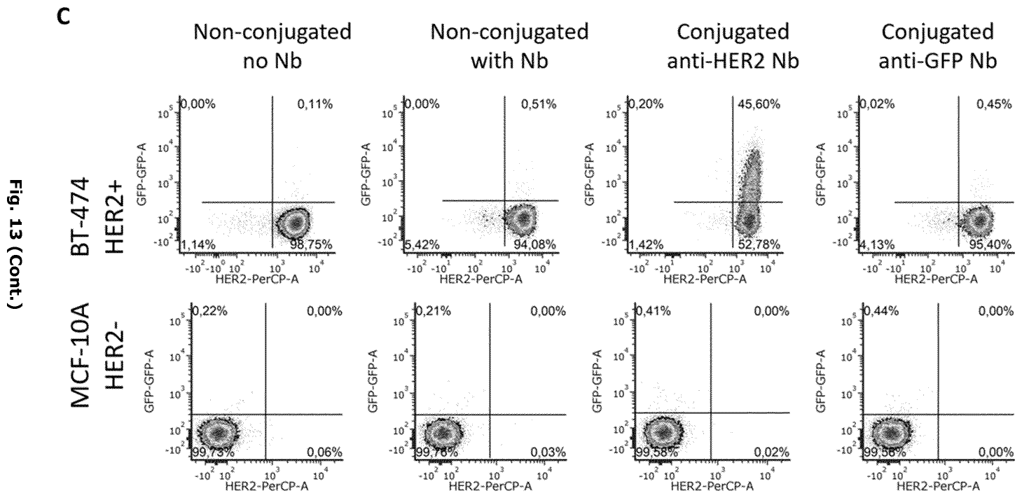

Irrespective of the presence or absence of nanobody, and irrespective of conjugation, the LPQTG-containing AAV vector (SEQ ID NO: 24) does not transduce the MCF-10A HER2-negative cells (FIGS. 13A, 13B, and 13C). This is most likely a result of the HBO mutations, as non-modified AAV2 does transduce MCF-10A cells efficiently (86%) at an MOI of 1E5 (data not shown).

For the BT-474 HER2-positive cells, the transduction efficiency is dramatically higher for AAV vectors conjugated with anti-HER2 nanobody relative to non-conjugated or anti-GFP nanobody-conjugated vectors.

These results clearly show that conjugation of our vectors with targeting nanobodies lead to increased transduction efficiency and specificity (FIGS. 13A, 13B, and 13C).

### Example 12: Comparison of Transduction Efficiencies

### Materials and Methods

LPQTG-containing AAV vectors (SEQ ID NO: 24 and SEQ ID NO: 30) [AAV2-HBO with an LPQTG tag located at position 587 with 3 GGSGS linkers flanking both sides and AAV9-A with an LPQTG tag located at position 455 with 3 GGSGS linkers flanking both sides] were incubated with Sortase A and anti-HER2 nanobody (SEQ ID NO: 12) (“Conjugated anti-HER2 Nb”), and with anti-HER2 nanobody (SEQ ID NO: 12) without Sortase A (“non-conjugated with Nb”) in 1× Sortase reaction buffer (300 mM Tris, 150 mM NaCl, 5 mM CaCl2, pH 7.5). The molar concentration of Sortase A was 10-fold higher than the molar concentration of AAV viral proteins in the mixture, while the nanobody was present in a 100-fold higher molar concentration relative to the AAV viral proteins. The reactions were incubated for 20 hours at 25*C before being dialyzed using a Micro Float-A-Lyzer 100 kDa MWCO (Repligen, F235049) against PBS to remove Sortase A enzyme and unreacted nanobody. Finally, the viral genome titer was determined using ddPCR.

BT-474 (HER2-positive) cells were cultured in MEBM (Lonza) supplemented with 10% FBS and RPMI1640 (Gibco) medium, supplemented Glutamax and 10% FBS, respectively. Cells were seeded at 50.000 cells in 90 ul of medium, and mixed with vector at an MOI of 1E5 vg/cell in 30 ul or an equivalent dilution of non-reacted nanobody only. This mixture was seeded in a 96-well plate, and after 3 h, 80 ul of medium was added. After 72 h, cells were washed with PBS, trypsinized, spun down, and re-suspended in 400 ul PBS with 1:10 000 PerCP-Cy5.5-conjugated anti-HER2 antibody (BioLegend 324416). After incubation for 30 min at RT, the cell suspension was analyzed using an Attune flow cytometer.

### Results and Discussion

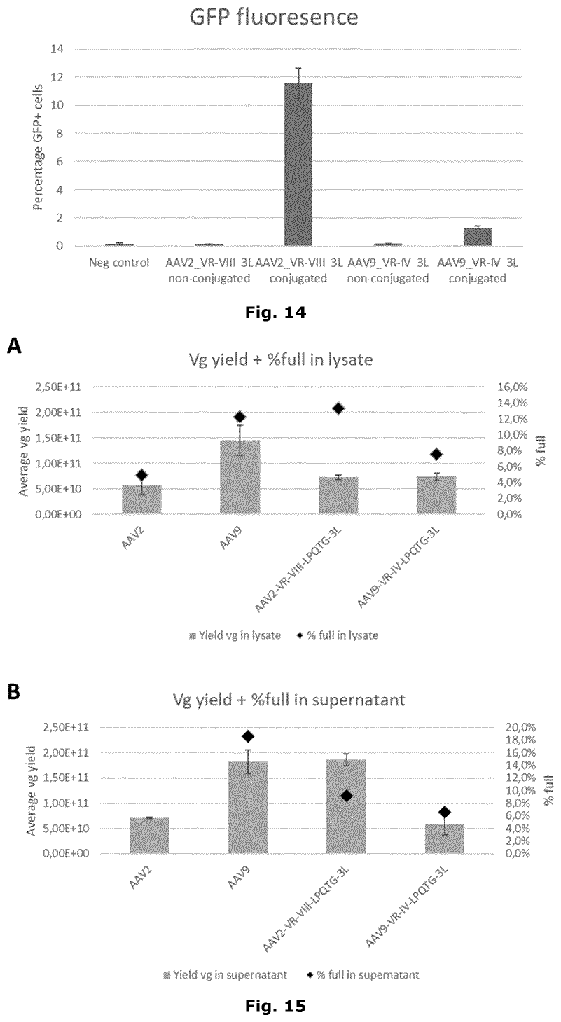

The transduction efficiency is 92-fold (AAV2_VR-VIII 3L, SEQ ID NO: 24) and 8-fold (AAV9_VR-IV 3L, SEQ ID NO: 30) higher for AAV vectors conjugated with anti-HER2 nanobody relative to non-conjugated vectors (FIG. 14).

These results clearly show that conjugation of our vectors with targeting nanobodies leads to increased transduction efficiency.

Interestingly, the effect is >10-fold more pronounced for AAV2_VR-VIII 3L, which has a lower conjugation efficiency (˜2%) compared to AAV9_VR-IV 3L (˜12%). This can be due to the insertion position or the base serotype that was used.

### Example 13: Comparison of Production Yields of Different Constructs

### Materials and Methods

HEK293 cells were seeded at 3.6E+06 viable cells per 10 cm petri dish in DMEM with 10% FBS and transfected using PEIpro following manufacturer's recommendations with a transgene plasmid (encoding ITR-flanked CAG-GFP), a plasmid coding for the adenoviral helper genes and a rep/cap-encoding plasmid. Each of AAV2, AAV9, AAV2-VR-VIII-LPQTG-3L [SEQ ID NO: 24] and AAV9-VR-IV-LPQTG-3L [SEQ ID NO: 30] were transfected in duplo. Transfected plates were stored at 37° C. and 5% CO2, and medium was exchanged for DMEM with 5% FBS 24 h after transfection. At 72 h after transfection, the supernatant was collected. Cells were dislodged using trypsin and stored in a separate falcon tube. Both tubes were spun at 1000 g for 5 min. The supernatant of the medium was transferred to a new tube, the supernatant of the cell fraction was discarded.

Next, the cell pellet was lysed using 3 freeze/thaw cycles, treated with a DNAse for 1 h at 37° C. and insoluble debris was finally removed using centrifugation. Using ddPCR, the viral genome (“Vg”) titer was determined from the supernatant and cell fraction. Using ELISA, the viral particle (“Vp”) titer was determined from the supernatant and cell fraction

### Results and Discussion

Although there is some variability in terms of production yield and percentage full, adherent production of the mutant vectors is similar to adherent production of the ancestral (AAV2 and AAV9) serotypes (FIGS. 15A and 15B). In other words, insertion of the LPQTG site with 15 amino acids flanking each side, does not result in a significant reduction of production yield.

### Sequences Referred to in the Application

AAV2 wild type Cap

| | |
|---|---|
| start VP2 | T138 |
| start VP3 | M203 |

VP1-VP2 transition P135-G141
PVKTAP [SEQ ID NO: 1]
VR-I S261-N268
SSQSGASN [SEQ ID NO: 2]
VR-IV S446-S474
SRTNT PSGTTTQSRL QFSQAGASDI RDQS [SEQ ID NO: 3]
T PSGTTTQS
[SEQ ID NO: 5]
VR-VIII Q575-V600
QYGSVS TNLQRGNRQA ATADVNTQGV [SEQ ID NO: 4]
LQRGNRQA A [SEQ ID NO: 6]
VP1 [SEQ ID NO: 7]

| | |
|---|---|
| 1 | MAADGYLPDW LEDTLSEGIR QWWKLKPGPP PPKPAERHKD DSRGLVLPGY |
| 51 | KYLGPFNGLD KGEPVNEADA AALEHDKAYD RQLDSGDNPY LKYNHADAEF |
| 101 | QERLKEDTSF GGNLGRAVFQ AKKRVLEPLG LVEEPVKTAP GKKRPVEHSP |
| 151 | VEPDSSSGTG KAGQQPARKR LNFGQTGDAD SVPDPQPLGQ PPAAPSGLGT |
| 201 | NTMATGSGAP MADNNEGADG VGNSSGNWHC DSTWMGDRVI TTSTRTWALP |
| 251 | TYNNHLYKQI SSQSGASNDN HYFGYSTPWG YFDFNRFHCH FSPRDWQRLI |
| 301 | NNNWGFRPKR LNFKLFNIQV KEVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFT FSYTFEDVPF HSSYAHSQSL DRLMNPLIDQ YLYYLSRTNT |
| 451 | PSGTTTQSRL QFSQAGASDI RDQSRNWLPG PCYRQQRVSK TSADNNNSEY |
| 501 | SWTGATKYHL NGRDSLVNPG PAMASHKDDE EKFFPQSGVL IFGKQGSEKT |
| 551 | NVDIEKVMIT DEEEIRTTNP VATEQYGSVS TNLQRGNRQA ATADVNTQGV |
| | A A |
| 601 | LPGMVWQDRD VYLQGPIWAK IPHTDGHFHP SPLMGGFGLK HPPPQILIKN |
| 651 | TPVPANPSTT FSAAKFASFI TQYSTGQVSV EIEWELQKEN SKRWNPEIQY |
| 701 | TSNYNKSVNV DFTVDINGVY SEPRPIGTRY LTRNL* |

VP2 [SEQ ID NO: 8]

| | |
|---|---|
| 138 | TAP GKKRPVEHSP |
| 151 | VEPDSSSGTG KAGQQPARKR LNFGQTGDAD SVPDPQPLGQ PPAAPSGLGT |
| 201 | NTMATGSGAP MADNNEGADG VGNSSGNWHC DSTWMGDRVI TTSTRTWALP |
| 251 | TYNNHLYKQI SSQSGASNDN HYFGYSTPWG YFDENRFHCH FSPRDWQRLI |
| 301 | NNNWGFRPKR LNFKLFNIQV KEVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFT FSYTFEDVPF HSSYAHSQSL DRLMNPLIDQ YLYYLSRTNT |
| 451 | PSGTTTQSRL QFSQAGASDI RDQSRNWLPG PCYRQQRVSK TSADNNNSEY |
| 501 | SWTGATKYHL NGRDSLVNPG PAMASHKDDE EKFFPQSGVL IFGKQGSEKT |
| 551 | NVDIEKVMIT DEEEIRTTNP VATEQYGSVS TNLQRGNRQA ATADVNTQGV |
| | A A |
| 601 | LPGMVWQDRD VYLQGPIWAK IPHTDGHFHP SPLMGGFGLK HPPPQILIKN |
| 651 | TPVPANPSTT FSAAKFASFI TQYSTGQVSV EIEWELQKEN SKRWNPEIQY |
| 701 | TSNYNKSVNV DFTVDTNGVY SEPRPIGTRY LTRNL* |

VP3 [SEQ ID NO: 9]

| | |
|---|---|
| 203 | MATGSGAP MADNNEGADG VGNSSGNWHC DSTWMGDRVI |
| 251 | TYNNHLYKQI SSQSGASNDN HYFGYSTPWG YFDENRFHCH FSPRDWQRLI |
| 301 | NNNWGFRPKR LNFKLFNIQV KEVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFT FSYTFEDVPF HSSYAHSQSL DRLMNPLIDQ YLYYLSRTNT |
| 451 | PSGTTTQSRL QFSQAGASDI RDQSRNWLPG PCYRQQRVSK TSADNNNSEY |
| 501 | SWTGATKYHL NGRDSLVNPG PAMASHKDDE EKFFPQSGVL IFGKQGSEKT |
| 551 | NVDIEKVMIT DEEEIRTTNP VATEQYGSVS TNLQRGNRQA ATADVNTQGV |
| | A A |
| 601 | LPGMVWQDRD VYLQGPIWAK IPHTDGHFHP SPLMGGFGLK HPPPQILIKN |
| 651 | TPVPANPSTT FSAAKFASFI TQYSTGQVSV EIEWELQKEN SKRWNPEIQY |
| 701 | TSNYNKSVNV DFTVDINGVY SEPRPIGTRY LTRNL* |

Mutations R585A and R588A refer to the HBº variant
Recognition motif:
Xn-LPXTG-Xm
LPXTG [SEQ ID NO: 10]

With X any natural AA, or wherein X is DEQN or wherein X is Q.
With n=0-20
With m=0-20

Surface exposed Lysines within 30 Angstrom vicinity of VR-I Loop are
K258 K321 K490 K507 K527 K532 K544 K549 K556 K620 K640 K649 K665 K692 K706

Surface exposed Lysines within 15 Angstrom vicinity of Loop VR-I are
K258 K507 K527 K549 K706

Surface exposed Lysines within 30 Angstrom vicinity of VR-IV Loop are
K258 K309 K313 K321 K490 K507 K527 K532 K544 K549 K556 K620 K640 K649 K665 K688 K692 K706

Surface exposed Lysines within 15 Angstrom vicinity of VR-IV Loop are:
K258 K490 K507 K532 K544 K549 K556 K665

Surface exposed Lysine within 5 Angstrom vicinity of VR-IV Loop is:
K549

Surface exposed Lysines within 30 Angstrom vicinity of VR-VIII Loop are:
K309 K490 K507 K527 K532 K544 K549 K556 K620 K640 K688 K706

Surface exposed Lysines within 15 Angstrom vicinity of VR-VIII Loop are:
K490 K507 K527 K532

Surface exposed Lysine within 5 Angstrom vicinity of VR-VIII Loop is:
K507

GFP nanobody:
GGGGSGGGGSGGGGSGGGGSEVQLVESGGALVQPGGSLRLSCAASGFPVNRYSMRWYRQAPGKEREWVAG
MSSAGDRSSYEDSVKGRFTISRDDARNTVYLQMNSLKPEDTAVYYCNVNVGFEYWGQGTQVTVSSAAADY
KDHDGDYKDHDIDYKDDDDKGAAHHHHHH [SEQ ID NO: 11]
HER2 nanobody:
GGGGSGGGGSGGGGSGGGGSEVQLVESGGGSVQAGGSLKLTCAASGYIFNSCGMGWYRQSPGRERELVSR
ISGDGDTWHKESVKGRFTISQDNVKKTLYLQMNSLKPEDTAVYFCAVCYNLETYWGQGTQVTVSSAAADY
KDHDGDYKDHDIDYKDDDDKGAAHHHHHH [SEQ ID NO: 12]
AAV2-VR-I-0L:
MAADGYLPDWLEDTLSEGIRQWWKLKPGPPPPKPAERHKDDSRGLVLPGYKYLGPENGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGLPQTGASNDNHYFGY
STPWGYFDFNRFHCHFSPRDWQRLINNNWGFRPKRLNFKLFNIQVKEVTQNDGTTTIANNLTSTVQVFTD
SEYQLPYVLGSAHQGCLPPFPADVFMVPQYGYLTLNNGSQAVGRSSFYCLEYFPSQMLRTGNNFTFSYTF
EDVPFHSSYAHSQSLDRLMNPLIDQYLYYLSRTNTPSGTTTQSRLQFSQAGASDIRDQSRNWLPGPCYRQ
QRVSKTSADNNNSEYSWTGATKYHLNGRDSLVNPGPAMASHKDDEEKFFPQSGVLIFGKQGSEKTNVDIE
KVMITDEEEIRTTNPVATEQYGSVSTNLQAGNAQAATADVNTQGVLPGMVWQDRDVYLQGPIWAKIPHTD
GHFHPSPLMGGFGLKHPPPQILIKNTPVPANPSTTFSAAKFASFITQYSTGQVSVEIEWELQKENSKRWN
PEIQYTSNYNKSVNVDFTVDINGVYSEPRPIGTRYLTRNL [SEQ ID NO: 13]
AAV2-VR-I-1L:
MAADGYLPDWLEDTLSEGIRQWWKLKPGPPPPKPAERHKDDSRGLVLPGYKYLGPENGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGGSGSGLPQTGGSGSG
ASNDNHYFGYSTPWGYFDFNRFHCHFSPRDWQRLINNNWGFRPKRLNFKLFNIQVKEVTQNDGTTTIANN
LTSTVQVFTDSEYQLPYVLGSAHQGCLPPFPADVFMVPQYGYLTLNNGSQAVGRSSFYCLEYFPSQMLRT
GNNFTFSYTFEDVPFHSSYAHSQSLDRLMNPLIDQYLYYLSRTNTPSGTTTQSRLQFSQAGASDIRDQSR
NWLPGPCYRQQRVSKTSADNNNSEYSWTGATKYHLNGRDSLVNPGPAMASHKDDEEKFFPQSGVLIFGKQ
GSEKTNVDIEKVMITDEEEIRTTNPVATEQYGSVSTNLQAGNAQAATADVNTQGVLPGMVWQDRDVYLQG
PIWAKIPHTDGHFHPSPLMGGFGLKHPPPQILIKNTPVPANPSTTFSAAKFASFITQYSTGQVSVEIEWE
LQKENSKRWNPEIQYTSNYNKSVNVDFTVDINGVYSEPRPIGTRYLTRNL [SEQ ID NO: 14]
AAV2-VR-I-2L:
MAADGYLPDWLEDTLSEGIRQWWKLKPGPPPPKPAERHKDDSRGLVLPGYKYLGPENGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGGSGSGGSGSGLPQTG
GSGSGGSGSGASNDNHYFGYSTPWGYFDFNRFHCHFSPRDWQRLINNNWGFRPKRLNFKLFNIQVKEVTQ
NDGTTTIANNLTSTVQVFTDSEYQLPYVLGSAHQGCLPPFPADVFMVPQYGYLTLNNGSQAVGRSSFYCL
EYFPSQMLRTGNNFTFSYTFEDVPFHSSYAHSQSLDRLMNPLIDQYLYYLSRTNTPSGTTTQSRLQFSQA
GASDIRDQSRNWLPGPCYRQQRVSKTSADNNNSEYSWTGATKYHLNGRDSLVNPGPAMASHKDDEEKFFP
QSGVLIFGKQGSEKTNVDIEKVMITDEEEIRTTNPVATEQYGSVSTNLQAGNAQAATADVNTQGVLPGMV
WQDRDVYLQGPIWAKIPHTDGHFHPSPLMGGFGLKHPPPQILIKNTPVPANPSTTFSAAKFASFITQYST
GQVSVEIEWELQKENSKRWNPEIQYTSNYNKSVNVDFTVDTNGVYSEPRPIGTRYLTRNL
[SEQ ID NO: 15]
AAV2-VR-IV-0L:
MAADGYLPDWLEDTLSEGIRQWWKLKPGPPPPKPAERHKDDSRGLVLPGYKYLGPENGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGASNDNHYFGYSTPWG
YFDFNRFHCHFSPRDWQRLINNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPSQMLRTGNNFTFSYTFEDVPF
HSSYAHSQSLDRLMNPLIDQYLYYLSRTNTPSGLPQTGTTTQSRLQFSQAGASDIRDQSRNWLPGPCYRQ
QRVSKTSADNNNSEYSWTGATKYHLNGRDSLVNPGPAMASHKDDEEKFFPQSGVLIFGKQGSEKTNVDIE
KVMITDEEEIRTTNPVATEQYGSVSTNLQAGNAQAATADVNTQGVLPGMVWQDRDVYLQGPIWAKIPHTD
GHFHPSPLMGGFGLKHPPPQILIKNTPVPANPSTTFSAAKFASFITQYSTGQVSVEIEWELQKENSKRWN
PEIQYTSNYNKSVNVDFTVDTNGVYSEPRPIGTRYLTRNL [SEQ ID NO: 16]
AAV2-VR-IV-1L:
MAADGYLPDWLEDTLSEGIRQWWKLKPGPPPPKPAERHKDDSRGLVLPGYKYLGPFNGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGASNDNHYFGYSTPWG
YFDFNRFHCHFSPRDWQRLINNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPSQMLRTGNNFTFSYTFEDVPF
HSSYAHSQSLDRLMNPLIDQYLYYLSRTNTPSGGGGGSGGGGSLPQTGGGGGSTTTQSRLQFSQAGASDI
RDQSRNWLPGPCYRQQRVSKTSADNNNSEYSWTGATKYHLNGRDSLVNPGPAMASHKDDEEKFFPQSGVL
IFGKQGSEKTNVDIEKVMITDEEEIRTTNPVATEQYGSVSTNLQAGNAQAATADVNTQGVLPGMVWQDRD
VYLQGPIWAKIPHTDGHFHPSPLMGGFGLKHPPPQILIKNTPVPANPSTTFSAAKFASFITQYSTGQVSV
EIEWELQKENSKRWNPEIQYTSNYNKSVNVDFTVDTNGVYSEPRPIGTRYLTRNL [SEQ ID NO: 17]
AAV2-VR-IV-3L:
MAADGYLPDWLEDTLSEGIRQWWKLKPGPPPPKPAERHKDDSRGLVLPGYKYLGPENGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGASNDNHYFGYSTPWG
YFDFNRFHCHFSPRDWQRLINNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPSQMLRTGNNFTFSYTFEDVPF
HSSYAHSQSLDRLMNPLIDQYLYYLSRTNTPSGGSGSGGSGSGGGGGSLPQTGGGGGSGSGSGGSGSGTT
TQSRLQFSQAGASDIRDQSRNWLPGPCYRQQRVSKTSADNNNSEYSWTGATKYHLNGRDSLVNPGPAMAS
HKDDEEKFFPQSGVLIFGKQGSEKTNVDIEKVMITDEEEIRTTNPVATEQYGSVSTNLQAGNAQAATADV
NTQGVLPGMVWQDRDVYLQGPIWAKIPHTDGHFHPSPLMGGFGLKHPPPQILIKNTPVPANPSTTFSAAK
FASFITQYSTGQVSVEIEWELQKENSKRWNPEIQYTSNYNKSVNVDFTVDTNGVYSEPRPIGTRYLTRNL
[SEQ ID NO: 18]
AAV2-VR-IV-4L:
MAADGYLPDWLEDTLSEGIRQWWKLKPGPPPPKPAERHKDDSRGLVLPGYKYLGPENGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGASNDNHYFGYSTPWG
YFDFNRFHCHFSPRDWQRLINNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPSQMLRTGNNFTFSYTFEDVPF
HSSYAHSQSLDRLMNPLIDQYLYYLSRTNTPSGGSGSGGSGSGGSGSGGGGGSLPQTGGGGGSGSGSGGS
GSGGSGSGTTTQSRLQFSQAGASDIRDQSRNWLPGPCYRQQRVSKTSADNNNSEYSWTGATKYHLNGRDS
LVNPGPAMASHKDDEEKFFPQSGVLIFGKQGSEKTNVDIEKVMITDEEEIRTTNPVATEQYGSVSTNLQA
GNAQAATADVNTQGVLPGMVWQDRDVYLQGPIWAKIPHTDGHFHPSPLMGGFGLKHPPPQILIKNTPVPA
NPSTTFSAAKFASFITQYSTGQVSVEIEWELQKENSKRWNPEIQYTSNYNKSVNVDFTVDINGVYSEPRP
IGTRYLTRNL [SEQ ID NO: 19]
AAV2-VR-IV-5L:
MAADGYLPDWLEDTLSEGIRQWWKLKPGPPPPKPAERHKDDSRGLVLPGYKYLGPFNGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGASNDNHYFGYSTPWG
YFDFNRFHCHFSPRDWQRLINNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPSQMLRTGNNFTFSYTFEDVPF
HSSYAHSQSLDRLMNPLIDQYLYYLSRTNTPSGGSGSGGSGSGGSGSGGSGSGGGGGSLPQTGGGGGSGS
GSGGSGSGGSGSGGSGSGTTTQSRLQFSQAGASDIRDQSRNWLPGPCYRQQRVSKTSADNNNSEYSWTGA
TKYHLNGRDSLVNPGPAMASHKDDEEKFFPQSGVLIFGKQGSEKTNVDIEKVMITDEEEIRTTNPVATEQ
YGSVSTNLQAGNAQAATADVNTQGVLPGMVWQDRDVYLQGPIWAKIPHTDGHFHPSPLMGGFGLKHPPPQ
ILIKNTPVPANPSTTFSAAKFASFITQYSTGQVSVEIEWELQKENSKRWNPEIQYTSNYNKSVNVDFTVD
TNGVYSEPRPIGTRYLTRNL [SEQ ID NO: 20]
AAV2-VR-VIII-0L:
MAADGYLPDWLEDTLSEGIRQWWKLKPGPPPPKPAERHKDDSRGLVLPGYKYLGPENGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGASNDNHYFGYSTPWG
YFDFNRFHCHFSPRDWQRLINNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPSQMLRTGNNFTFSYTFEDVPF
HSSYAHSQSLDRLMNPLIDQYLYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGVLIFGKQGSEKTNVDIEKVMIT
DEEEIRTTNPVATEQYGSVSTNLQAGNLPQTGAQAATADVNTQGVLPGMVWQDRDVYLQGPIWAKIPHTD
GHFHPSPLMGGFGLKHPPPQILIKNTPVPANPSTTFSAAKFASFITQYSTGQVSVEIEWELQKENSKRWN
PEIQYTSNYNKSVNVDFTVDTNGVYSEPRPIGTRYLTRNL [SEQ ID NO: 21]
AAV2-VR-VIII-1L:
MAADGYLPDWLEDTLSEGIRQWWKLKPGPPPPKPAERHKDDSRGLVLPGYKYLGPENGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGASNDNHYFGYSTPWG
YFDFNRFHCHFSPRDWQRLINNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPSQMLRTGNNFTFSYTFEDVPF
HSSYAHSQSLDRLMNPLIDQYLYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGVLIFGKQGSEKTNVDIEKVMIT
DEEEIRTTNPVATEQYGSVSTNLQAGNGGGGSLPQTGGGGGSAQAATADVNTQGVLPGMVWQDRDVYLQG
PIWAKIPHTDGHFHPSPLMGGFGLKHPPPQILIKNTPVPANPSTTFSAAKFASFITQYSTGQVSVEIEWE
LOKENSKRWNPEIQYTSNYNKSVNVDFTVDTNGVYSEPRPIGTRYLTRNL [SEQ ID NO: 22]
AAV2-VR-VIII-2L:
MAADGYLPDWLEDTLSEGIRQWWKLKPGPPPPKPAERHKDDSRGLVLPGYKYLGPENGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGASNDNHYFGYSTPWG
YFDFNRFHCHFSPRDWQRLINNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPSQMLRTGNNFTFSYTFEDVPF
HSSYAHSQSLDRLMNPLIDQYLYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGVLIFGKQGSEKTNVDIEKVMIT
DEEEIRTTNPVATEQYGSVSTNLQAGNGSGSGGGGGSLPQTGGGGGSGSGSGAQAATADVNTQGVLPGMV
WQDRDVYLQGPIWAKIPHTDGHFHPSPLMGGFGLKHPPPQILIKNTPVPANPSTTFSAAKFASFITQYST
GQVSVEIEWELQKENSKRWNPEIQYTSNYNKSVNVDFTVDTNGVYSEPRPIGTRYLTRNL
[SEQ ID NO: 23]
AAV2-VR-VIII-3L:
MAADGYLPDWLEDTLSEGIRQWWKLKPGPPPPKPAERHKDDSRGLVLPGYKYLGPENGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGASNDNHYFGYSTPWG
YFDFNRFHCHESPRDWQRLINNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPSQMLRTGNNFTFSYTFEDVPF
HSSYAHSQSLDRLMNPLIDQYLYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGVLIFGKQGSEKTNVDIEKVMIT
DEEEIRTTNPVATEQYGSVSTNLQAGNGSGSGGSGSGGGGGSLPQTGGGGGSGSGSGGSGSGAQAATADV
NTQGVLPGMVWQDRDVYLQGPIWAKIPHTDGHFHPSPLMGGFGLKHPPPQILIKNTPVPANPSTTESAAK
FASFITQYSTGQVSVEIEWELQKENSKRWNPEIQYTSNYNKSVNVDFTVDINGVYSEPRPIGTRYLTRNL
[SEQ ID NO: 24]
AAV2-VR-VIII-4L:
MAADGYLPDWLEDTLSEGIRQWWKLKPGPPPPKPAERHKDDSRGLVLPGYKYLGPENGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGASNDNHYFGYSTPWG
YFDFNRFHCHFSPRDWQRLINNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPSQMLRTGNNFTFSYTFEDVPF
HSSYAHSQSLDRLMNPLIDQYLYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGVLIFGKQGSEKTNVDIEKVMIT
DEEEIRTTNPVATEQYGSVSTNLQAGNGSGSGGSGSGGSGSGGGGGSLPQTGGGGGSGSGSGGSGSGGSG
SGAQAATADVNTQGVLPGMVWQDRDVYLQGPIWAKIPHTDGHFHPSPLMGGFGLKHPPPQILIKNTPVPA
NPSTTFSAAKFASFITQYSTGQVSVEIEWELQKENSKRWNPEIQYTSNYNKSVNVDFTVDTNGVYSEPRP
IGTRYLTRNL [SEQ ID NO: 25]
AAV2-VR-VIII-5L:
MAADGYLPDWLEDTLSEGIRQWWKLKPGPPPPKPAERHKDDSRGLVLPGYKYLGPENGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGASNDNHYFGYSTPWG
YFDFNRFHCHFSPRDWQRLINNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPSQMLRTGNNFTFSYTFEDVPF
HSSYAHSQSLDRLMNPLIDQYLYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGVLIFGKQGSEKTNVDIEKVMIT
DEEEIRTTNPVATEQYGSVSTNLQAGNGSGSGGSGSGGSGSGGSGSGGGGGSLPQTGGGGGSGSGSGGSG
SGGSGSGGSGSGAQAATADVNTQGVLPGMVWQDRDVYLQGPIWAKIPHTDGHFHPSPLMGGFGLKHPPPQ
ILIKNTPVPANPSTTFSAAKFASFITQYSTGQVSVEIEWELQKENSKRWNPEIQYTSNYNKSVNVDFTVD
TNGVYSEPRPIGTRYLTRNL [SEQ ID NO: 26]
AAV9-VR-IV-0L:
MAADGYLPDWLEDNLSEGIREWWALKPGAPQPKANQQHQDNARGLVLPGYKYLGPGNGLDKGEPVNAADA
AALEHDKAYDQQLKAGDNPYLKYNHADAEFQERLKEDTSFGGNLGRAVFQAKKRLLEPLGLVEEAAKTAP
GKKRPVEQSPQEPDSSAGIGKSGAQPAKKRLNFGQTGDTESVPDPQPIGEPPAAPSGVGSLTMASGGGAP
VADNNEGADGVGSSSGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYFPSQMLRTGNNFQFSYEFENV
PFHSSYAHSQSLDRLMNPLIDQYLYYLSKTINGSGLPQTGQNQQTLKFSVAGPSAMAVQGRNYIPGPSYR
QQRVSTTVTQNNNSEFAWPGASSWALNGRNSLMNPGPAMASHKEGEDRFFPLSGSLIFGKQGTGRDNVDA
DKVMITNEEEIKTTNPVATESYGQVATNHQSAQAQAQTGWVQNQGILPGMVWQDRDVYLQGPIWAKIPHT
DGNFHPSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL [SEQ ID NO: 27]
AAV9-VR-IV-1L:
MAADGYLPDWLEDNLSEGIREWWALKPGAPQPKANQQHQDNARGLVLPGYKYLGPGNGLDKGEPVNAADA
AALEHDKAYDQQLKAGDNPYLKYNHADAEFQERLKEDTSFGGNLGRAVFQAKKRLLEPLGLVEEAAKTAP
GKKRPVEQSPQEPDSSAGIGKSGAQPAKKRLNFGQTGDTESVPDPQPIGEPPAAPSGVGSLTMASGGGAP
VADNNEGADGVGSSSGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYFPSQMLRTGNNFQFSYEFENV
PFHSSYAHSQSLDRLMNPLIDQYLYYLSKTINGSGGSGSGLPQTGGSGSGQNQQTLKFSVAGPSAMAVQG
RNYIPGPSYRQQRVSTTVTQNNNSEFAWPGASSWALNGRNSLMNPGPAMASHKEGEDRFFPLSGSLIFGK
QGTGRDNVDADKVMITNEEEIKTTNPVATESYGQVATNHQSAQAQAQTGWVQNQGILPGMVWQDRDVYLQ
GPIWAKIPHTDGNFHPSPLMGGFGMKHPPPQILIKNTPVPADPPTAFNKDKLNSFITQYSTGQVSVEIEW
ELQKENSKRWNPEIQYTSNYYKSNNVEFAVNTEGVYSEPRPIGTRYLTRNL [SEQ ID NO: 28]
AAV9-VR-IV-2L:
MAADGYLPDWLEDNLSEGIREWWALKPGAPQPKANQQHQDNARGLVLPGYKYLGPGNGLDKGEPVNAADA
AALEHDKAYDQQLKAGDNPYLKYNHADAEFQERLKEDTSFGGNLGRAVFQAKKRLLEPLGLVEEAAKTAP
GKKRPVEQSPQEPDSSAGIGKSGAQPAKKRLNFGQTGDTESVPDPQPIGEPPAAPSGVGSLTMASGGGAP
VADNNEGADGVGSSSGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYFPSQMLRTGNNFQFSYEFENV
PFHSSYAHSQSLDRLMNPLIDQYLYYLSKTINGSGGSGSGGSGSGLPQTGGSGSGGSGSGQNQQTLKFSV
AGPSAMAVQGRNYIPGPSYRQQRVSTTVTQNNNSEFAWPGASSWALNGRNSLMNPGPAMASHKEGEDRFF
PLSGSLIFGKQGTGRDNVDADKVMITNEEEIKTTNPVATESYGQVATNHQSAQAQAQTGWVQNQGILPGM
VWQDRDVYLQGPIWAKIPHTDGNFHPSPLMGGFGMKHPPPQILIKNTPVPADPPTAFNKDKLNSFITQYS
TGQVSVEIEWELQKENSKRWNPEIQYTSNYYKSNNVEFAVNTEGVYSEPRPIGTRYLTRNL
[SEQ ID NO: 29]
AAV9-VR-IV-3L:
MAADGYLPDWLEDNLSEGIREWWALKPGAPQPKANQQHQDNARGLVLPGYKYLGPGNGLDKGEPVNAADA
AALEHDKAYDQQLKAGDNPYLKYNHADAEFQERLKEDTSFGGNLGRAVFQAKKRLLEPLGLVEEAAKTAP
GKKRPVEQSPQEPDSSAGIGKSGAQPAKKRLNFGQTGDTESVPDPQPIGEPPAAPSGVGSLTMASGGGAP
VADNNEGADGVGSSSGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYFPSQMLRTGNNFQFSYEFENV
PFHSSYAHSQSLDRLMNPLIDQYLYYLSKTINGSGGSGSGGSGSGGGGGSLPQTGGGGGSGSGSGGSGSG
QNQQTLKFSVAGPSAMAVQGRNYIPGPSYRQQRVSTTVTQNNNSEFAWPGASSWALNGRNSLMNPGPAMA
SHKEGEDRFFPLSGSLIFGKQGTGRDNVDADKVMITNEEEIKTTNPVATESYGQVATNHQSAQAQAQTGW
VQNQGILPGMVWQDRDVYLQGPIWAKIPHTDGNFHPSPLMGGFGMKHPPPQILIKNTPVPADPPTAFNKD
KLNSFITQYSTGQVSVEIEWELQKENSKRWNPEIQYTSNYYKSNNVEFAVNTEGVYSEPRPIGTRYLTRN
L [SEQ ID NO: 30]
AAV9-VR-VIII-0L:
MAADGYLPDWLEDNLSEGIREWWALKPGAPQPKANQQHQDNARGLVLPGYKYLGPGNGLDKGEPVNAADA
AALEHDKAYDQQLKAGDNPYLKYNHADAEFQERLKEDTSFGGNLGRAVFQAKKRLLEPLGLVEEAAKTAP
GKKRPVEQSPQEPDSSAGIGKSGAQPAKKRLNFGQTGDTESVPDPQPIGEPPAAPSGVGSLTMASGGGAP
VADNNEGADGVGSSSGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYFPSQMLRTGNNFQFSYEFENV
PFHSSYAHSQSLDRLMNPLIDQYLYYLSKTINGSGQNQQTLKFSVAGPSAMAVQGRNYIPGPSYRQQRVS
TTVTQNNNSEFAWPGASSWALNGRNSLMNPGPAMASHKEGEDRFFPLSGSLIFGKQGTGRDNVDADKVMI
TNEEEIKTTNPVATESYGQVATNHQSAQALPQTGQAQTGWVQNQGILPGMVWQDRDVYLQGPIWAKIPHT
DGNFHPSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL [SEQ ID NO: 31]
AAV9-VR-VIII-1L:
MAADGYLPDWLEDNLSEGIREWWALKPGAPQPKANQQHQDNARGLVLPGYKYLGPGNGLDKGEPVNAADA
AALEHDKAYDQQLKAGDNPYLKYNHADAEFQERLKEDTSFGGNLGRAVFQAKKRLLEPLGLVEEAAKTAP
GKKRPVEQSPQEPDSSAGIGKSGAQPAKKRLNFGQTGDTESVPDPQPIGEPPAAPSGVGSLTMASGGGAP
VADNNEGADGVGSSSGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYFPSQMLRTGNNFQFSYEFENV
PFHSSYAHSQSLDRLMNPLIDQYLYYLSKTINGSGQNQQTLKFSVAGPSAMAVQGRNYIPGPSYRQQRVS
TTVTQNNNSEFAWPGASSWALNGRNSLMNPGPAMASHKEGEDRFFPLSGSLIFGKQGTGRDNVDADKVMI
TNEEEIKTTNPVATESYGQVATNHQSAQAGSGSGLPQTGGSGSGQAQTGWVQNQGILPGMVWQDRDVYLQ
GPIWAKIPHTDGNFHPSPLMGGFGMKHPPPQILIKNTPVPADPPTAFNKDKLNSFITQYSTGQVSVEIEW
ELQKENSKRWNPEIQYTSNYYKSNNVEFAVNTEGVYSEPRPIGTRYLTRNL [SEQ ID NO: 32]
AAV9-VR-VIII-3L:
MAADGYLPDWLEDNLSEGIREWWALKPGAPQPKANQQHQDNARGLVLPGYKYLGPGNGLDKGEPVNAADA
AALEHDKAYDQQLKAGDNPYLKYNHADAEFQERLKEDTSFGGNLGRAVFQAKKRLLEPLGLVEEAAKTAP
GKKRPVEQSPQEPDSSAGIGKSGAQPAKKRLNFGQTGDTESVPDPQPIGEPPAAPSGVGSLTMASGGGAP
VADNNEGADGVGSSSGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYFPSQMLRTGNNFQFSYEFENV
PFHSSYAHSQSLDRLMNPLIDQYLYYLSKTINGSGQNQQTLKFSVAGPSAMAVQGRNYIPGPSYRQQRVS
TTVTQNNNSEFAWPGASSWALNGRNSLMNPGPAMASHKEGEDRFFPLSGSLIFGKQGTGRDNVDADKVMI
TNEEEIKTTNPVATESYGQVATNHQSAQAGSGSGGSGSGGGGGSLPQTGGGGGSGSGSGGSGSGQAQTGW
VQNQGILPGMVWQDRDVYLQGPIWAKIPHTDGNFHPSPLMGGFGMKHPPPQILIKNTPVPADPPTAFNKD
KLNSFITQYSTGQVSVEIEWELQKENSKRWNPEIQYTSNYYKSNNVEFAVNTEGVYSEPRPIGTRYLTRN
L [SEQ ID NO: 33]
AAV2-VR-VIII-LPETG-3L:
MAADGYLPDWLEDTLSEGIRQWWKLKPGPPPPKPAERHKDDSRGLVLPGYKYLGPFNGLDKGEPVNEADA
AALEHDKAYDRQLDSGDNPYLKYNHADAEFQERLKEDTSFGGNLGRAVFQAKKRVLEPLGLVEEPVKTAP
GKKRPVEHSPVEPDSSSGTGKAGQQPARKRLNFGQTGDADSVPDPQPLGQPPAAPSGLGTNTMATGSGAP
MADNNEGADGVGNSSGNWHCDSTWMGDRVITTSTRTWALPTYNNHLYKQISSQSGASNDNHYFGYSTPWG
YFDFNRFHCHFSPRDWQRLINNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPSQMLRTGNNFTFSYTFEDVPF
HSSYAHSQSLDRLMNPLIDQYLYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGVLIFGKQGSEKTNVDIEKVMIT
DEEEIRTTNPVATEQYGSVSTNLQAGNGSGSGGSGSGGGGGSLPETGGGGGSGSGSGGSGSGAQAATADV
NTQGVLPGMVWQDRDVYLQGPIWAKIPHTDGHFHPSPLMGGFGLKHPPPQILIKNTPVPANPSTTFSAAK
FASFITQYSTGQVSVEIEWELQKENSKRWNPEIQYTSNYNKSVNVDFTVDTNGVYSEPRPIGTRYLTRNL
[SEQ ID NO: 34]
AAV9-VR-IV-LPETG-3L:
MAADGYLPDWLEDNLSEGIREWWALKPGAPQPKANQQHQDNARGLVLPGYKYLGPGNGLDKGEPVNAADA
AALEHDKAYDQQLKAGDNPYLKYNHADAEFQERLKEDTSFGGNLGRAVFQAKKRLLEPLGLVEEAAKTAP
GKKRPVEQSPQEPDSSAGIGKSGAQPAKKRLNFGQTGDTESVPDPQPIGEPPAAPSGVGSLTMASGGGAP
VADNNEGADGVGSSSGNWHCDSQWLGDRVITTSTRTWALPTYNNHLYKQISNSTSGGSSNDNAYFGYSTP
WGYFDFNRFHCHFSPRDWQRLINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYFPSQMLRTGNNFQFSYEFENV
PFHSSYAHSQSLDRLMNPLIDQYLYYLSKTINGSGGSGSGGSGSGGGGGSLPETGGGGGSGSGSGGSGSG
QNQQTLKFSVAGPSAMAVQGRNYIPGPSYRQQRVSTTVTQNNNSEFAWPGASSWALNGRNSLMNPGPAMA
SHKEGEDRFFPLSGSLIFGKQGTGRDNVDADKVMITNEEEIKTTNPVATESYGQVATNHQSAQAQAQTGW
VQNQGILPGMVWQDRDVYLQGPIWAKIPHTDGNFHPSPLMGGFGMKHPPPQILIKNTPVPADPPTAFNKD
KLNSFITQYSTGQVSVEIEWELQKENSKRWNPEIQYTSNYYKSNNVEFAVNTEGVYSEPRPIGTRYLTRN
L [SEQ ID NO: 35]
VP1 capsid VR-IV region amino acids 453-454
NTPSGLPQTGTTTQS [SEQ ID NO: 36]
VP1 capsid VR-VIII region amino acids 587-588
LQRGNGGSGSLPQTGGGSGSRQAAT [SEQ ID NO: 37]
VP1 capsid VR-VIII region amino acids 587-588 double linker
LQRGNGGSGSGGSGSLPQTGGGSGSGGSGSRQAAT [SEQ ID NO: 38]
Linker sequence 1: GGGGS [SEQ ID NO: 39]
Xn-NPXTG-Xm: NPXTG [SEQ ID NO: 40]
Xn-LPXTA-Xm: LPXTA [SEQ ID NO: 41]
Xn-LAXTG-Xm: LAXTG [SEQ ID NO: 42]
Sortase A recognition sequence 1: LPQTG [SEQ ID NO: 43]
Sortase A recognition sequence 2: LPETG [SEQ ID NO: 44]
Sortase A recognition sequence 3: LPNTG [SEQ ID NO: 45]
Sortase A recognition sequence 4: LPDTG [SEQ ID NO: 46]
Linker sequence 2: GGSGS [SEQ ID NO: 47]
Xn-LPXAG-Xm: LPXAG [SEQ ID NO: 48]
Xn-LPXLG-Xm: LPXLG [SEQ ID NO: 49]
Xn-APXTG-Xm: APXTG [SEQ ID NO: 50]
Xn-LPXSG-Xm: LPXSG [SEQ ID NO: 51]
Xn-FPXTG-Xm: FPXTG [SEQ ID NO: 52]
Xn-XPKTG-Xm: XPKTG [SEQ ID NO: 53]
Xn-LPEXG-Xm: LPEXG [SEQ ID NO: 54]
Xn-NPQTN-Xm: NPQTN [SEQ ID NO: 55]
Xn-QVPTG-Xm: QVPTG [SEQ ID NO: 56]
Xn-LPNTA-Xm: LPNTA [SEQ ID NO: 57]
Xn-LPLTG-Xm: LPLTG [SEQ ID NO: 58]
Xn-APKTG-Xm: APKTG [SEQ ID NO: 59]
Xn-DPKTG-Xm DPKTG [SEQ ID NO: 60]
Xn-SPKTG-Xm: SPKTG [SEQ ID NO: 61]
Xn-APATG-Xm: APATG [SEQ ID NO: 62]
Xn-LAETG-Xm: LAETG [SEQ ID NO: 63]
Xn-LPEAG-Xm: LPEAG [SEQ ID NO: 64]
Xn-LPECG-Xm: LPECG [SEQ ID NO: 65]
Xn-LPESG-Xm: LPESG [SEQ ID NO: 66]
Xn-LMVGG-Xm: LMVGG [SEQ ID NO: 67]

1. An adeno-assisted virus (AAV) capsid protein characterized in that in one or more of the VP1-VP2 transition region, VR-I region, VR-IV region or VR-VIII region a sortase recognition sequence is inserted.

2. The AAV capsid protein according to claim 1, characterized in that the inserted sortase recognition sequence is a sequence having a general sequence motif selected from the group consisting of: Xn-LPXTG-Xm [SEQ ID NO: 10], Xn-NPXTG-Xm [SEQ ID NO: 40], Xn-LPXTA-Xm [SEQ ID NO: 41], Xn-LAXTG-Xm [SEQ ID NO: 42], Xn-LPXAG-Xm [SEQ ID NO: 48], Xn-LPXLG-Xm [SEQ ID NO: 49], Xn-APXTG-Xm [SEQ ID NO: 50], Xn-LPXSG-Xm [SEQ ID NO: 51], Xn-FPXTG-Xm [SEQ ID NO: 52], Xn-XPKTG-Xm, [SEQ ID NO: 53], and Xn-LPEXG-Xm, [SEQ ID NO: 54], wherein n and m range from 0 to 25, and wherein X is any natural amino acid.

3. The AAV capsid protein according to claim 1, characterized in that the inserted sortase recognition sequence has the general sequence motif Xn-LPXTG-Xm [SEQ ID NO: 10].

4. The AAV capsid protein according to claim 1, wherein n and/or m range from 0 to 20.

5. The AAV capsid protein according to claim 2, wherein X is Glutamine or Glutamic acid.

6. The capsid protein according to claim 1, wherein the VP1-VP2 transition region is defined by PVKTAP [SEQ ID NO: 1], the VR-I region is defined by SSQSGASN [SEQ ID NO: 2], the VR-IV region is defined by SRTNTPSGTTTQSRLQFSQAGASDIRDQS [SEQ ID NO: 3], and/or the VR-VIII region is defined by QYGSVSTNLQRGNRQAATADVNTQGV [SEQ ID NO: 4] in AAV2 or a corresponding amino acid sequence in another AAV serotype.

7. The capsid protein according to claim 1, wherein the insertion in the VR-IV region is in the fragment with TPSGTTTQS [SEQ ID NO: 5], and/or in that the insertion in the VR-VIII region is in the fragment with LQRGNRQAA [SEQ ID NO: 6] in AAV2 or a corresponding amino acid sequence in another AAV serotype.

8. The capsid protein according to claim 1, wherein in a region wherein a sortase recognition sequence LPXTG [SEQ ID NO: 10] is inserted, one or more amino acids of said region are deleted, or substituted.

9. The capsid protein according to claim 1, wherein the AAV is AAV2, or AAV9.

10. The capsid protein according to claim 1, wherein n is between 5 and 20, preferably between 5 and 15, more preferably between 10 and 15.

11. The capsid protein according to claim 1, wherein m is between 5 and 20, preferably between 5 and 15, more preferably between 10 and 15.

12. The capsid protein according to claim 1, wherein the linker sequence Xn or Xm consists of at least 80% of Glycine, Serine, Threonine and Alanine.

13. The capsid protein according to claim 1, wherein the linker sequence Xm or Xn consists of amino acids selected from Glycine, Serine, Threonine and Alanine.

14. The capsid protein according to claim 1, wherein X in LPXTG [SEQ ID NO: 10] is Aspartic acid, Glutamic acid, Asparagine or Glutamine, preferably wherein X in LPXTG [SEQ ID NO: 10] is Glutamine.

15. The capsid protein according to claim 1, wherein one or more Lysines (K) of the AAV capsid protein are mutated into Glycine (Gly), Serine (Ser), or Alanine (Ala), preferably Glycine (Gly).

16. A nucleic acid encoding the capsid protein of any one of claim 1.

17. An expression vector comprising the nucleic acid of claim 16.

18. An AAV particle comprising an AAV capsid protein according to claim 1.

19. A conjugated AAV particle comprising an AAV capsid protein characterized by a remnant sortase recognition sequence in the VP1-VP2 transition region, VR-I region, VR-IV region and/or VR-VIII region, and wherein the remnant sortase recognition sequence is operably linked to a heterologous conjugate molecule.

20. The conjugated AAV particle according to claim 19, wherein the ratio of unconjugated capsid AAV protein over conjugated AAV capsid protein is between 1/59 and 59/1, preferably between 1/20 and 20/1, preferably between 1/10 and 10/1, more preferably between 1/5 and 5/1.

21. The conjugated AAV particle according to claim 19, which is operably linked via the remnant sortase recognition sequence to a heterologous conjugate molecule characterized by the presence of a terminal triglycine amino acid sequence.

22. The conjugated AAV particle according to claim 19, which is operably linked via the remnant sortase recognition sequence to a targeting moiety.

23. A conjugated AAV particle according to claim 19 for use as a medicament.

24. A method of producing a conjugated AAV particle, comprising the steps of
contacting cells with one or more nucleic acids encoding a AAV capsid proteins wherein one or more of the VP1-VP2 transition region, VR-I region, VR-IV region or VR-VIII region comprise a sortase recognition sequence;
allowing the cell to assemble the plurality of AAV capsid proteins into an AAV particle and collecting said AAV particles therefrom; and
contacting the AAV particles with a sortase and a heterologous conjugate molecule.


SEQUENCE LISTING (67 sequences)

SEQ ID NO: 1 - protein, 6 aa, synthetic construct
PVKTAP

SEQ ID NO: 2 - protein, 8 aa, synthetic construct
SSQSGASN

SEQ ID NO: 3 - protein, 29 aa, synthetic construct
SRTNTPSGTTTQSRLQFSQAGASDIRDQS

SEQ ID NO: 4 - protein, 26 aa, synthetic construct
QYGSVSTNLQRGNRQAATADVNTQGV

SEQ ID NO: 5 - protein, 9 aa, synthetic construct
TPSGTTTQS

SEQ ID NO: 6 - protein, 9 aa, synthetic construct
LQRGNRQAA

SEQ ID NO: 7 - protein, 735 aa, synthetic construct [sequence omitted]

SEQ ID NO: 8 - protein, 598 aa, synthetic construct
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - protein, 533 aa, synthetic construct
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 10 - protein, 5 aa, synthetic construct
LPXTG

SEQ ID NO: 11 - protein, 169 aa, synthetic construct
GGGGSGGGGSGGGGSGGGGSEVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSAAADYKDHDGDYKDHDIDYKDDDDKGAAHHHHHH

SEQ ID NO: 12 - protein, 169 aa, synthetic construct
GGGGSGGGGSGGGGSGGGGSEVQLVESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSSAAADYKDHDGDYKDHDIDYKDDDDKGAAHHHHHH

SEQ ID NO: 13 - protein, 740 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGLPQTGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 14 - protein, 750 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGGSGSGLPQTGGSGSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 15 - protein, 750 aa, synthetic construct; identical to SEQ ID NO: 14

SEQ ID NO: 16 - protein, 740 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGLPQTGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 17 - protein, 755 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGGGGGSGGGGSLPQTGGGGGSTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 18 - protein, 770 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGGSGSGGSGSGGGGGSLPQTGGGGGSGSGSGGSGSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 19 - protein, 780 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGGSGSGGSGSGGSGSGGGGGSLPQTGGGGGSGSGSGGSGSGGSGSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 20 - protein, 790 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGGSGSGGSGSGGSGSGGSGSGGGGGSLPQTGGGGGSGSGSGGSGSGGSGSGGSGSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 21 - protein, 740 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNLPQTGAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 22 - protein, 750 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGGGGSLPQTGGGGGSAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 23 - protein, 760 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGSGSGGGGGSLPQTGGGGGSGSGSGAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 24 - protein, 770 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGSGSGGSGSGGGGGSLPQTGGGGGSGSGSGGSGSGAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 25 - protein, 780 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGSGSGGSGSGGSGSGGGGGSLPQTGGGGGSGSGSGGSGSGGSGSGAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 26 - protein, 790 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGSGSGGSGSGGSGSGGSGSGGGGGSLPQTGGGGGSGSGSGGSGSGGSGSGGSGSGAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 27 - protein, 741 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGLPQTGQNQQTLKFSVAGPSAMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 28 - protein, 751 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGGSGSGLPQTGGSGSGQNQQTLKFSVAGPSAMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - protein, 761 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGGSGSGGSGSGLPQTGGSGSGGSGSGQNQQTLKFSVAGPSAMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - protein, 771 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGGSGSGGSGSGGGGGSLPQTGGGGGSGSGSGGSGSGQNQQTLKFSVAGPSAMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 31 - protein, 741 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSAMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQALPQTGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - protein, 751 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSAMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAGSGSGLPQTGGSGSGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - protein, 771 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSAMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAGSGSGGSGSGGGGGSLPQTGGGGGSGSGSGGSGSGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 34 - protein, 770 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGSGSGGSGSGGGGGSLPETGGGGGSGSGSGGSGSGAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 35 - protein, 771 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGGSGSGGSGSGGGGGSLPETGGGGGSGSGSGGSGSGQNQQTLKFSVAGPSAMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 36 - protein, 15 aa, synthetic construct
NTPSGLPQTGTTTQS

SEQ ID NO: 37 - protein, 25 aa, synthetic construct
LQRGNGGSGSLPQTGGGSGSRQAAT

SEQ ID NO: 38 - protein, 35 aa, synthetic construct
LQRGNGGSGSGGSGSLPQTGGGSGSGGSGSRQAAT

SEQ ID NO: 39 - protein, 5 aa, synthetic construct
GGGGS

SEQ ID NO: 40 - protein, 5 aa, synthetic construct
NPXTG

SEQ ID NO: 41 - protein, 5 aa, synthetic construct
LPXTA

SEQ ID NO: 42 - protein, 5 aa, synthetic construct
LAXTG

SEQ ID NO: 43 - protein, 5 aa, synthetic construct
LPQTG

SEQ ID NO: 44 - protein, 5 aa, synthetic construct
LPETG

SEQ ID NO: 45 - protein, 5 aa, synthetic construct
LPNTG

SEQ ID NO: 46 - protein, 5 aa, synthetic construct
LPDTG

SEQ ID NO: 47 - protein, 5 aa, synthetic construct
GGSGS

SEQ ID NO: 48 - protein, 5 aa, synthetic construct
LPXAG

SEQ ID NO: 49 - protein, 5 aa, synthetic construct
LPXLG

SEQ ID NO: 50 - protein, 5 aa, synthetic construct
APXTG

SEQ ID NO: 51 - protein, 5 aa, synthetic construct
LPXSG

SEQ ID NO: 52 - protein, 5 aa, synthetic construct
FPXTG

SEQ ID NO: 53 - protein, 5 aa, synthetic construct
XPKTG

SEQ ID NO: 54 - protein, 5 aa, synthetic construct
LPEXG

SEQ ID NO: 55 - protein, 5 aa, synthetic construct
NPQTN

SEQ ID NO: 56 - protein, 5 aa, synthetic construct
QVPTG

SEQ ID NO: 57 - protein, 5 aa, synthetic construct
LPNTA

SEQ ID NO: 58 - protein, 5 aa, synthetic construct
LPLTG

SEQ ID NO: 59 - protein, 5 aa, synthetic construct
APKTG

SEQ ID NO: 60 - protein, 5 aa, synthetic construct
DPKTG

SEQ ID NO: 61 - protein, 5 aa, synthetic construct
SPKTG

SEQ ID NO: 62 - protein, 5 aa, synthetic construct
APATG

SEQ ID NO: 63 - protein, 5 aa, synthetic construct
LAETG

SEQ ID NO: 64 - protein, 5 aa, synthetic construct
LPEAG

SEQ ID NO: 65 - protein, 5 aa, synthetic construct
LPECG

SEQ ID NO: 66 - protein, 5 aa, synthetic construct
LPESG

SEQ ID NO: 67 - protein, 5 aa, synthetic construct
LMVGG